(19) United States
(12) Patent Application Publication
(10) Pub. No.: US 2022/0056393 A1
(43) Pub. Date: Feb. 24, 2022

(54) METHODS AND SYSTEMS FOR INTRACELLULAR DELIVERY

(71) Applicant: CELLFE, INC., Alameda, CA (US)

(72) Inventors: Sewoon HAN, Albany, CA (US); Alexander ALEXEEV, Atlanta, GA (US); Alla ZAMARAYEVA, Berkeley, CA (US); Todd SULCHEK, Atlanta, GA (US)

(73) Assignee: CELLFE, INC., Alameda, CA (US)

(21) Appl. No.: 17/521,237

(22) Filed: Nov. 8, 2021

Related U.S. Application Data
(63) Continuation of application No. 17/339,757, filed on Jun. 4, 2021.
Application No. 17/339,757 is a continuation of application No. PCT/US2019/064310, filed on Dec. 3, 2019.
(60) Provisional application No. 62/775,351, filed on Dec. 4, 2018.

(51) Int. Cl. C12M 1/42; C12M 3/06; C12M 1/00

(52) U.S. Cl. CPC C12M 35/04; C12M 47/04; C12M 23/16

(57) ABSTRACT

The present disclosure provides systems and methods for intracellular delivery. The systems and methods may comprise the use of a cell processing apparatus which may comprise a plurality of compression elements such as ridges. The intracellular delivery may be caused by rapid compression of cells, which may result in a reduction of a cell volume. The compression may occur while the cells pass through gaps formed by at least a subset of the ridges. The cell processing apparatus may further comprise one or more recovery spaces which are positioned between adjacent ridges. The cells may recover at least a portion of the reduced cell volume by absorbing media and/or reagents surrounding the cells while flowing through the recovery spaces. The ridges may also divert less compressible cells into a diversion channel, thereby sorting the cells based on various cell properties and/or preventing clogging within the apparatus.

### CROSS-REFERENCE

This application claims the benefit of U.S. Provisional Patent Application 62/775,351, filed on Dec. 4, 2018, which is incorporated herein by reference in its entirety for all purposes.

### BACKGROUND

Intracellular delivery can be used in many different applications, such as gene transfection, editing, cell labeling, and cell interrogation. However, conventional delivery methods, such as microinjection, electroporation, and sonoporation, may have low delivery efficiencies, especially for large molecules, such as molecules with a size of at least 2000 kilodaltons (kDa), and large particles, such as particles with the size of at least 100 nanometers.

### SUMMARY

Provided are cell processing apparatuses and systems as well as methods of using these systems and producing intracellular delivery. The intracellular delivery may be caused by rapid compression of cells, resulting in a volume loss. The subsequent recovery of the cells may be performed in a media, comprising reagents. The cells may recover and increase their volume by absorbing the media and the reagents. The cells may be suspended in the media and flow through a cell processing apparatus, which may comprise a plurality of compressive elements such as ridges. The compression may occur when the cells pass through gaps formed by the compressive elements (e.g., ridges), which may be smaller than the cell size. The compressive elements (e.g., ridges) may divert less compressible cells into a channel, thereby sorting the cells based on their compressibility, and/or size, and/or preventing clogging of the apparatus. This approach may allow processing of cells with varying sizes including large cells (such as cells with a size of at least about 20 micrometers in diameter) with high delivery efficiencies.

An aspect of the present disclosure provides a cell processing apparatus comprising: a first wall comprising a first surface, wherein the first wall extends along a flow direction; a second wall comprising a second surface, wherein the second wall extends along the flow direction; a plurality of ridges connected to the first wall, wherein the plurality of ridges extends from the first surface toward the second surface, and wherein a ridge of the plurality of ridges comprises a ridge surface that forms a gap with the second surface; and a diversion channel extending along the flow direction, which diversion channel is at least partially defined by at least a subset of the plurality of ridges.

In some embodiments, the cell processing apparatus further comprises two or more outlets, wherein at least one of the two or more outlets is aligned with the diversion channel, and wherein at least an additional one of the two or more outlets is positioned away from the diversion channel. In some embodiments, the cell processing apparatus further comprises an intermediate outlet fluidically coupled and open to the diversion channel and disposed between a pair of the plurality of ridges. In some embodiments, the cell processing apparatus further comprises side walls each connected to at least one of the first wall and the second wall, wherein each of the plurality of ridges extends between the side walls at an angle between 10° and 80° relative to the flow direction. In some embodiments, all ridges of the plurality of ridges are parallel to one another. In some embodiments, at least two ridges of the plurality of ridges are oriented at different angles relative to the flow direction. In some embodiments, each of the plurality of ridges has a ridge thickness of between 5 micrometers and 100 micrometers. In some embodiments, the ridge surface of the ridge of the plurality of ridges is parallel to the second surface. In some embodiments, the plurality of ridges each has a ridge surface that forms a gap with the second wall, and a height of the gaps varies along the flow direction. In some embodiments, the plurality of ridges comprises a first ridge set and a second ridge set, and the first ridge set and the second ridge set form a chevron pattern. In some embodiments, the first ridge set and/or the second ridge set comprises a plurality of leading edges, positioned at different distances from one of the side walls. In some embodiments, the diversion channel is positioned between the first ridge set and the second ridge set. In some embodiments, the first ridge set and the second ridge set overlap and are offset relative to each other along the flow direction, thereby forming a tortuous path of the diversion channel. In some embodiments, a first ridge of the plurality of ridges comprises a first ridge surface which forms a first gap with the second surface, and a second ridge of the plurality of ridges comprises a second ridge surface which forms a second gap with the second surface, and the first gap has a different height than the second gap. In some embodiments, a height of the gap is adjustable. In some embodiments, at least one of the side walls is flexible such that the first wall is movable relative to the second wall. In some embodiments, a first ridge of the plurality of ridges comprises a first ridge surface which forms a first gap with the second surface, and a second ridge of the plurality of ridges comprises a second ridge surface which forms a second gap with the second surface, and the first gap has a different width than the second gap. In some embodiments, the cell processing apparatus further comprises a recovery space positioned between two adjacent ridges of the plurality of ridges, wherein a distance of the recovery space, along the flow direction, is between 100 micrometers and 1000 micrometers. In some embodiments, a width of the diversion channel, measured perpendicular to the flow direction, is variable along the flow direction. In some embodiments, the cell processing apparatus further comprises an additional diversion channel, wherein the diversion channel is positioned between each of the plurality of ridges and one of the side walls, and wherein the additional diversion channel is positioned between each of the plurality of ridges and an additional one of the side walls.

Another aspect of the present disclosure provides a system for cell processing, the system comprising: a cell processing apparatus comprising: a first wall comprising a first surface; a second wall comprising a second surface; a plurality of ridges connected to the first wall, wherein the plurality of ridges extends from the first surface toward the second surface, and wherein a ridge of the plurality of ridges comprises a ridge surface that forms a gap with the second surface; and a diversion channel extending along a flow direction of the cell processing apparatus, which diversion channel is defined at least partially by at least a subset of the plurality of ridges; a pressure source fluidically coupled to the cell processing apparatus; one or more sensors operably coupled to the cell processing apparatus; and a system controller, operably coupled to the pressure source and to the one or more sensors, and configured to control an operation of the pressure source based on one or more inputs from the one or more sensors.

In some embodiments, the system further comprises a temperature controlling module, thermally coupled to at least one of the first wall and the second wall, and operably coupled to the system controller, wherein the temperature controlling module is configured to control a temperature of a medium flown through the cell processing apparatus. In some embodiments, the one or more sensors comprise a cell counter positioned in the diversion channel. In some embodiments, the system further compries a gap adjuster, mechanically coupled to at least one of the first wall and the second wall, and operably coupled to the system controller, wherein the gap adjuster is configured to adjust a height of the gap between the ridge surface and the second surface. In some embodiments, the one or more inputs comprise at least one of: a pressure inside the cell processing apparatus, a cell count at an inlet of the cell processing apparatus, a cell count at an outlet of the cell processing apparatus, a temperature inside the cell processing apparatus, a flow rate inside the cell processing apparatus, an optical image from the cell processing apparatus, and a position of the first wall relative to the second wall. In some embodiments, the system further comprises an additional cell processing apparatus comprising an additional plurality of ridges, wherein a ridge of the additional plurality of ridges comprises an additional ridge surface which forms an additional gap with an additional second surface of an additional second wall of the additional cell processing apparatus, and wherein the additional gap has a different height than the gap; and a cell sorter positioned upstream and fluidically coupled to the cell processing apparatus and the additional cell processing apparatus, such that the cell processing apparatus and the second cell processing apparatus are configured to operate in parallel. In some embodiments, the system further comprises an additional cell processing apparatus, wherein the cell processing apparatus and the additional cell processing apparatus are connected in sequence. In some embodiments, the system further comprises an inlet fluidically coupled to and positioned between the cell processing apparatus and the additional cell processing apparatus. In some embodiments, the system further comprises two or more outlets, wherein: at least one of the two or more outlets is aligned with the diversion channel, and at least an additional one of the two or more outlets is positioned away from the diversion channel. In some embodiments, the system further comprises side walls each connected to at least one of the first wall and the second wall, wherein each of the plurality of ridges extends between the side walls of the cell processing apparatus at an angle between 10° and 80° relative to the flow direction. In some embodiments, the pressure source is a pump.

Another aspect of the present disclosure provides a cell processing method comprising: (a) directing cells into a cell processing apparatus comprising: a plurality of ridges, wherein a ridge of the plurality of ridges comprises a ridge surface that forms a gap with a surface of the cell processing apparatus, and wherein the gap is configured to reduce a cell volume of cells flowing therethrough, a recovery space between two adjacent ridges of the plurality of ridges, which recovery space is configured to recover at least a portion of the cell volume reduced by the gap, and a diversion channel extending along a flow direction of the cell processing apparatus, which diversion channel is defined at least partially by at least a subset of the plurality of ridges; (b) flowing a first subset of the cells through the gap and the recovery space to generate one or more processed cells, wherein the first subset of the cells has a reduced cell volume upon flowing through the gap, and wherein the reduced cell volume is recovered at least partially by absorbing a surrounding medium upon the first subset of the cells flowing through the recovery space, thereby generating the one or more processed cells; and (c) directing a second subset of the cells not passing through the gap or the recovery space into the diversion channel.

In some embodiments, an average duration of the first subset of the cells passing through the gap is less than about 1 second. In some embodiments, cells of the first subset of cells have an average volume reduction of at least about 10%, as compared to an original cell volume. In some embodiments, the cell processing method further comprises, sorting the cells prior to (a). In some embodiments, the cell processing method further comprises, sorting the cells after (b). In some embodiments, the cell processing method further comprises flowing the first subset of cells through one or more gaps formed by one or more ridges of the plurality of ridges, thereby resulting in one or more volume reductions of the first subset of the cells. In some embodiments, a linear flow rate of the cells flowing through the cell processing apparatus is adjustable. In some embodiments, the linear flow rate is adjusted between 10% and 50% of an average linear flow rate with a frequency between 0.1 Hertz (Hz) and 100 Hz. In some embodiments, the linear flow rate is adjusted in response to an input received from one or more sensors. In some embodiments, the cell processing method further comprises reversing a direction of fluid flow of the cells through the cell processing apparatus. In some embodiments, a flow rate of the cells flowing through the cell processing apparatus is between about 5 millimeters per second and 200 millimeters per second. In some embodiments, (a) comprises vibrating the cell processing apparatus. In some embodiments, the cell processing apparatus is vibrated with an amplitude of greater than 1 micrometer. In some embodiments, the cell processing apparatus is vibrated with a frequency of greater than 1 Hz.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A is a cross-sectional side view of a cell processing apparatus, illustrating two ridges, used for cell compression, in accordance with some examples.

FIG. 1B is a cross-sectional top view of the cell processing apparatus in FIG. 1A, illustrating the same two ridges, positioned at an angle relative to the flow direction and forming a diversion channel along one of the side walls, in accordance with some examples.

FIG. 1C is a cross-sectional top view of a cell processing apparatus, illustrating different angles formed by ridges with the principal axis, in accordance with some examples.

FIG. 1D, FIG. 1E, FIG. 1F, FIG. 1G, FIG. 1H, FIG. 1I, FIG. 1J, and FIG. 1K are cross-sectional side views of a portion of a cell processing apparatus, illustrating different shapes of ridges, in accordance with some examples.

FIG. 2A is a cross-sectional top view of a cell processing apparatus, illustrating two diversion channels, extending along each of the side walls, in accordance with some examples.

FIG. 2B is a cross-sectional top view of a cell processing apparatus, illustrating a diversion channel increasing in width along the flow direction, in accordance with some examples.

FIG. 2C is a cross-sectional top view of a cell processing apparatus, illustrating a diversion channel decreasing in width along the flow direction, in accordance with some examples.

FIG. 2D is a cross-sectional top view of a cell processing apparatus, illustrating a straight diversion channel, positioned away from the side walls and along the center axis, in accordance with some examples.

FIG. 2E is a cross-sectional top view of a cell processing apparatus, illustrating a curved diversion channel formed by overlapping ridges, positioned away from the side walls and along the center axis, in accordance with some examples.

FIG. 2F is a cross-sectional top view of a cell processing apparatus, illustrating an initial diversion channel splitting, downstream, into two channels and changing the position within the interior of the cell processing apparatus, in accordance with some examples.

FIG. 2G is a cross-sectional top view of a cell processing apparatus, illustrating a diversion channel, changing the position from one side wall to another side wall within the interior of the cell processing apparatus, in accordance with some examples.

FIG. 3A is a cross-sectional top view of a cell processing apparatus, illustrating ridges having leading edges offset relative to each other along the principal axis, in accordance with some examples.

FIGS. 3B and 3C are cross-sectional top views of two examples of a cell processing apparatus, illustrating different shapes of ridges.

FIG. 3D is a cross-sectional top view of a cell processing apparatus, illustrating curved side walls, in accordance with some examples.

FIG. 4A is a cross-sectional side view of a cell processing apparatus, illustrating different gaps formed by two ridges, in accordance with some examples.

FIGS. 4B and 4C are cross-sectional front views of a cell processing apparatus, illustrating different gap heights formed by the same ridge at different operating stages, in accordance with some examples.

FIG. 4D is a cross-sectional side view of a cell processing apparatus, illustrating recovery spaces with different interior heights, in accordance with some examples.

FIG. 4E is a cross-sectional side view of a cell processing apparatus, illustrating a channel with ridges attached to opposite walls, in accordance with some examples.

FIGS. 5A and 5B are cross-sectional front views of a cell processing apparatus, illustrating different gap widths formed by ridges, in accordance with some examples.

FIGS. 6A and 6B are cross-sectional front views of a cell processing apparatus, illustrating different side channel heights, corresponding to two different ridges, in accordance with some examples.

FIG. 7A is a schematic representation of a system, comprising a cell processing apparatus and various other components, coupled to the cell processing apparatus, in accordance with some examples.

FIG. 7B is a schematic representation of a system, comprising a cell sorter, coupled to three cell processing apparatuses, in accordance with some examples.

FIG. 7C is a schematic representation of a system, comprising three cell processing apparatuses, connected in a sequence, in accordance with some examples.

FIG. 7D is a schematic representation of a system, comprising three cell processing apparatuses, operating in parallel, in accordance with some examples.

FIG. 8 is a process flowchart corresponding to a method of intracellular delivery, in accordance with some examples.

FIG. 9A is a cross-sectional side view of a cell processing apparatus, illustrating a core-shell type of molecules, processed in the apparatus, in accordance with some examples.

FIG. 9B is a cross-sectional side view of a two-phase droplet generator, illustrating forming a core-shell types of molecules, in accordance with some examples.

FIG. 10 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Provided herein are methods and systems for intracellular delivery. The methods may comprise providing a fluidic device (e.g., a microfluidic device). The methods and systems may facilitate delivery of one or more reagents or substances (such as therapeutic reagents, gene-editing reagents) into cells. The methods and systems may comprise the use of a fluidic device. The fluidic device may comprise one or more compression elements.

The fluidic device may comprise a plurality of channels (e.g., microchannels). A plurality of the channels may be one or more microchannels. The number of the channels in the fluidic device may be at least 1, 2, 3, 4, 5, 6, 7, 8, 9, 10, 11, 12, 13, 14, 15, 16, 17, 18, 19, 20, or more channels. Increasing the number of channels in the fluidic device may increase an exit flow rate or the throughput of a process. Two or more fluidic channels may be connected in parallel, in series, or a combination of series and parallel. The channels may be connected by various configurations.

The fluidic device may comprise a principal axis. The principal axis may be parallel to a flow direction of the device. The principal axis may be parallel to the plurality of microchannels. The method may further comprise subjecting one or more cells to flow through the microchannel of the fluidic device. As the cells flow through the microchannel, the cells may be in contact with the compressive element comprised in the microchannel. The microchannel may have a cross-sectional dimension that is greater than or equal to about 1 micrometers (μm), 5 μm, 10 μm, 15 μm, 20 μm, 30 μm, 40 μm, 50 μm, 60 μm, 70 μm, 80 μm, 90 μm, 100 μm, 150 μm, 200 μm, 250 μm, 300 μm, 350 μm, 400 μm, 450 μm, 500 μm, 600 μm, 700 μm, 800 μm, 900 μm, 1,000 μm, or more. In some cases, the cross-sectional dimension of the channel may be less than or equal to about 2,000 μm, 1,500 μm, 1,000 μm, 850 μm, 700 μm, 550 μm, 400 μm, 300 μm, 200 μm, 100 μm, 80 μm, 60 μm, 40 μm, 20 μm, 10 μm, or less. In some cases, the cross-sectional dimension of the channel may fall within any of the two values described above, e.g., between about 20 μm and about 1,000 μm, or between about 50 μm and about 100 μm.

The microchannel may comprise a plurality of compressive elements. The plurality of compressive elements may be one or more compressive elements. Each microchannel in the microfluidic device may include one or more compressive elements. The plurality of compressive elements may comprise ridges. The compressive elements may comprise compressive surfaces. Compressive surfaces may have different shapes and/or curvatures, such as rectangular, triangular, cylindrical, spherical, or other shapes and/or curvatures. The compressive elements may have different sizes, such as different surface areas. As a cell flows through the fluidic device, the cell may be in contact with the compressive element. The compressive element may result in a cell volume reduction. After the compression, the cell may recover part or all of its reduced volume by absorbing media surrounding the cell.

The compression element may comprise a plurality of compressive surfaces, e.g., greater than or equal to about 2, 3, 4, 5, 6, 7, 8, 9, 10, 11, 12, 13, 14, 15, 16, 17, 18, 19, 20 compressive surfaces, or more. The compressive surfaces may be ridges. The compressive surfaces may or may not extend parallel with respect to one another. In some cases, at least a subset of the compressive surfaces extends parallel with respect to one another. The compressive surfaces may have regular or irregular cross-sectional shapes. In some cases, the compressive surfaces have rectangular cross-sections.

Dimensions of the compressive surfaces may vary, depending upon various factors, such as cell flow rate, cell type, cell size, cell stiffness, cell adhesiveness, substance type, channel material and/or channel size. For example, in some cases, the compressive surfaces have an average width that is greater than or equal to about 1 μm, 5 μm, 10 μm, 15 μm, 20 μm, 30 μm, 40 μm, 50 μm, 60 μm, 70 μm, 80 μm, 90 μm, 100 μm, 150 μm, 200 μm, 250 μm, 300 μm, 350 μm, 400 μm, 450 μm, 500 μm, or more. In some cases, the compressive surfaces have an average width that is less than or equal to about 800 μm, 700 μm, 600 μm, 500 μm, 400 μm, 300 μm, 200 μm, 150 μm, 100 μm, 80 μm, 60 μm, 40 μm, 20 μm, or less. In some cases, the compressive surfaces have an average width that falls between any of the two values described above, for example, between about 20 μm and 250 μm.

So that the cells may pass through the channel, the compressive element may have a dimension (e.g., a height) that is smaller than a cross-sectional dimension of the channel. Consequently, there may be a gap between a surface the compressive element (e.g., a ridge surface) and an interior surface of the channel or the device. The gap may have a size that is adjustable. The gap size may be adjusted based upon a variety of factors, such as cell size, cell type, cell stiffness, cell adhesiveness, flow rate, channel material, channel size, temperature, substance type, and/or substance size. In some cases, the gap size may be greater than or equal to about 0.1 μm, 0.5 μm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 22 μm, 24 μm, 26 μm, 28 μm, 30 μm, or more. In some cases, the gap size may be less than or equal to about 50 μm, 45 μm, 40 μm, 35 μm, 30 μm, 25 μm, 20 μm, 18 μm, 16 μm, 14 μm, 12 μm, 10 μm, 8 μm, 6 μm, 4 μm, 2 μm, 1 μm, or less. In some cases, the gap size may fall within a range of any of the two values described above, for example, between about 1 μm and about 20 μm, or between about 3 μm and 15 μm.

The gap size may be smaller than a cell size. For example, the gap size may be less than or equal to about 50%, 45%, 40%, 35%, 30%, 25%, 20%, 15%, 10% of an average diameter of a cell, or less. In some cases, the gap size may be less than or equal to about 50%, 45%, 40%, 35%, 30%, 25%, 20%, 15%, 10% of a diameter of a given cell comprised in the cells that pass through the channel.

In cases where multiple compressive elements (e.g., compressive surfaces) are comprised in the channel, each compressive element may have the same or a different dimension. As a result, gap sizes between each compressive element and an interior surface of the channel may or may not differ. In some cases, at least a subset (e.g., at least about 5%, 10%, 15%, 20%, 30%, 35%, 40%, 45%, 50%, or more) of the compressive elements have different dimensions.

The compressive elements may be spaced apart from one another. Such configuration may facilitate periodic compression and expansion of the cells. For example, as a cell passes through the channel, the cell may be compressed while in contact with a compressive element. Following the compression and prior to being subjected to contact with a subsequent compressive element, the cell may flow into an area (e.g., a recovery space) between two adjacent compressive elements where the cell may expand and recover some or all of the volume lost during the compression. Dimensions or shapes of spaces between each pair of adjacent compressive elements may or may not be the same. In some cases, the compressive elements are equally distant. In some cases, a space between each pair of adjacent compressive elements progressively increases or decreases along a flow direction of the cells. The flow direction may be the main flow direction of a majority of the cells. The flow direction may be in alignment with a principal axis of the channel. The flow direction may be a direction from an inlet of the channel to an outlet of the channel.

The compressive element may comprise an angled compressive element, such as an angled surface relative to the principal axis of the microfluidic device. All compressive elements in the microfluidic device may be parallel to one another (i.e., oriented at the same angle relative to the principal axis of the device). Alternatively, different compressive elements may have different angles relative to the principal axis of the device. In some examples, the angles may be from 10 to 50 degrees, or from 20 to 80 degrees, or from 30 to 60 degrees. For example, the angles may by greater than or equal to about 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, 75 degrees, 80 degrees, 85 degrees, or more. Angled compressive elements may facilitate the removal of unwanted substances from the microchannel. The unwanted substances may comprise, for example, nonviable cells, aggregates, clogging agents, excess contrasting agents, excess amounts of other reagents, or more. The removal of unwanted substances may contribute to increasing the throughput and/or efficiency of cell processing.

A plurality of cells may be directed to pass through the fluidic device. The plurality of cells may comprise greater than or equal to about 20 million, 50 million, 100 million, 200 million, 300 million, 400 million, 500 million, 600 million, 700 million, 800 million cells, or more. In some cases, the plurality of cells may be less than or equal to about 2,000 million, 1,500 million, 1,000 million, 800 million, 600 million, 400 million, 200 million, 100 million cells, or less.

In some examples, the methods may comprise rapid compression of cells which may result in a volume loss by the cells. The compression may occur when the cells pass through the fluidic device comprising the compression elements (e.g., ridges). The compression may be rapid. The compression may occur within a short time period. For example, the compression may occur in less than or equal to about 2 seconds (s), 1.8 s, 1.6 s, 1.4 s, 1.2 s, 1 s, 900 milliseconds (ms), 800 ms, 700 ms, 600 ms, 500 ms, 400 ms, 350 ms, 300 ms, 280 ms, 260 ms, 240 ms, 220 ms, 200 ms, 180 ms, 160 ms, 140 ms, 120 ms, 100 ms, 90 ms, 80 ms, 70 ms, 60 ms, 50 ms, 40 ms, 30 ms, 20 ms, 10 ms, 5 ms, 1 ms, or less. In some cases, the compression may occur within a time period that falls between any of the two values described above, for example, between about 10 ms and about 300 ms. As an example, the compression may occur in less than 1 second, such as within 10 microseconds to 300 milliseconds. The compression time may depend on the flowrate, cell size, compressive element (e.g., ridge) geometry, and various other factors, such as the factors further described above or elsewhere herein.

During the compression, the cells may change their volume (e.g., experience volume loss of at least 10% or even at least 30% of the pre-compression volume) rather than simply changing their shapes (e.g., adapting to the compression gap without significant volume change). A combination of the compression speed and the volume loss may distinguish the methods and systems of the present disclosure from conventional microfluidic techniques, in which cells may simply be reshaped. In some examples, the cells may change shapes and without substantially changing the volume (e.g., less than or equal to about 25%, 20%, 18%, 16%, 14%, 12%, 10%, 8%, 6%, 5%, 4%, 3%, 2% volume change relative to the original volume, or less). In other examples, the cells may change their volume without substantially changing their shapes. In some examples, both cell shape (e.g., morphology) and cell volume may change.

The volume reduction (or loss) may be temporary. The compression may cause a cell to lose at least about 10%, 15%, 20%, 25%, 30%, 35%, 40%, 45%, 50% of its volume, or more. The compressed state may be a non-natural state for the cells and the cells may attempt to recover to the original volume. As a result, the compression may be followed by cell expansion and recovery. During the recovery, the cells may increase their volume by absorbing media surrounding the cells. The media which comprise various reagents as described above or elsewhere herein.

The cells may attempt to recover to their original volume as soon as no further compression forces are applied on them. In some cases, the methods of the present disclosure may comprise releasing the cells into a recovery space after compression by the compressive elements. In some cases, the cells may be immediately released into the recovery space. The recovery space may be inside the same cell processing apparatus. The recovery process may allow the cells to recover at least partially and to increase the volume by absorbing the surrounding media. The media may comprise one or more reagents, which may be introduced into the cells as a part of this recovery process. Examples of such reagents may comprise plasmids and magnetic nanoparticles (e.g., introduced into stem cells or other types of cells for various applications) and mRNA (e.g., introduced into primary peripheral blood mononuclear cells or other types of cells for various applications). Other reagents and cell types may be used.

As provided herein, one or more reagents may be directed into an interior of cells including large cells. The reagents may comprise large molecules. In some examples, the plurality of substances may have an average molecular weight greater than or equal to about 0.5 megadaltons (MDa), 0.6 MDa, 0.7 MDa, 0.8 MDa, 0.9 MDa, 1.0 MDa, 1.1 MDa, 1.2 MDa, 1.3 MDa, 1.4 MDa, 1.5 MDa, 1.6 MDa, 1.7 MDa, 1.8 MDa, 1.9 MDa, 2.0 MDa, 2.1 MDa, 2.2 MDa, 2.3 MDa, 2.4 MDa, 2.5 MDa, 2.6 MDa, 2.7 MDa, 2.8 MDa, 2.9 MDa, 3.0 MDa, 3.5 MDa, 4.0 MDa, 4.5 MDa, 5.0 MDa, or more. In some cases, each of the substances may has a molecular weight that is greater than or equal to about 0.5 megadaltons (MDa), 0.6 MDa, 0.7 MDa, 0.8 MDa, 0.9 MDa, 1.0 MDa, 1.1 MDa, 1.2 MDa, 1.3 MDa, 1.4 MDa, 1.5 MDa, 1.6 MDa, 1.7 MDa, 1.8 MDa, 1.9 MDa, 2.0 MDa, 2.1 MDa, 2.2 MDa, 2.3 MDa, 2.4 MDa, 2.5 MDa, 2.6 MDa, 2.7 MDa, 2.8 MDa, 2.9 MDa, 3.0 MDa, 3.5 MDa, 4.0 MDa, 4.5 MDa, 5.0 MDa, or more.

The reagents may or may not comprise a charged substance. For example, the reagents may comprise a drug, a nucleic acid molecule, an antigen, a polypeptide, an antibody, an antigen, a hapten, an enzyme, or combinations thereof. The nucleic acid molecule may comprise deoxyribonucleic acid (DNA), ribonucleic acid (RNA), peptide nucleic acid (PNA), or combinations thereof.

In some examples, the reagents comprise reagents greater than or equal to about 5 kilobases (kB), 6 kB, 7 kB, 8 kB, 9 kB, 10 kB, 11 kB, 12 kB, 13 kB, 14 kB, 15 kB, 16 kB, 17 kB, 18 kB, 19 kB, 20 kB, 21 kB, 22 kB, 23 kb, 24 kB, 25 kB, 26 kB, 27 kB, 28 kB, 29 kB, 30 kB, or larger. One example reagent may be DNA plasmids with size larger than 10 kB.

In some cases, large reagents, such as reagents at least about 10 kB in size may not be introduced by conventional microfluidic methods, such as squeezing cells in narrow pores to improve the membrane poration characteristics, followed by the slow diffusion of reagents through the temporary membrane pores. Since the diffusion is slower for larger reagents, such reagents may not be effectively delivered using these diffusion methods. Furthermore, the degree of cell membrane poration, proposed by conventional methods, may be limited to ensure cell viability and avoid cell damage or death. In some examples, conventional microfluidic devices for intracellular delivery may be prone to clogging because narrow channels may be used for achieving high membrane shear and opening membrane pores. Increasing flowrates, e.g., to reduce clogging, may result in cell damage.

Referring to apparatuses and methods described herein, in some examples, a sequence of compression (volume reduction) and recovery (reagent absorption) may be performed multiple times, e.g., once for each compressive element (e.g., compression ridge) provided inside a cell processing apparatus. Furthermore, in some examples, this processing sequence may be repeated in a different manner, e.g., using a different gap height (level of compression), a different compressive element geometry, a different gap length (compression duration), a different flowrate (compression and recovery duration), different reagents and/or reagents concentrations. For example, the ridges with different gaps and/or different widths and profiles may affect the degree, speed, and/or duration of cell compressions. Furthermore, in some examples, a cell processing apparatus may comprise a diversion channel for removal of less compressible cells. For example, the compressive elements (e.g., ridges) may be angled (not perpendicular) relative to the flow direction, which may be referred to as diagonal ridge orientation. A diversion channel may be positioned at the end of these ridges, e.g., along one of the side walls and/or away from the side walls. Cells, which are not sufficiently compressible and cannot pass through the gap formed by these compressive elements (e.g., ridges), may be pushed (by the flow) along these compressive elements and into the diversion channel. In some examples, these cells may then be collected separately from cells that have undergone one or more compression-recovery sequences.

In general, the methods and systems described herein may be used to deliver a variety of reagents (e.g., macromolecules) to a variety of different cell types. In some cases, the intracellular delivery may be achieved with high throughput and minimal clogging, while posing lower risk of cell death and aggregation than conventional methods. In some cases, the substances may be delivered into the plurality of cells at an efficiency of greater than or equal to about 30%, 40%, 50%, 60%, 70%, 80%, 90%, 95%, or more. The efficiency of the method may be higher than diffusion-based methods and/or endocytosis. The efficiency of the method may be maintained while increasing the throughput and/or rate of cell processing.

### Cell Processing Apparatus

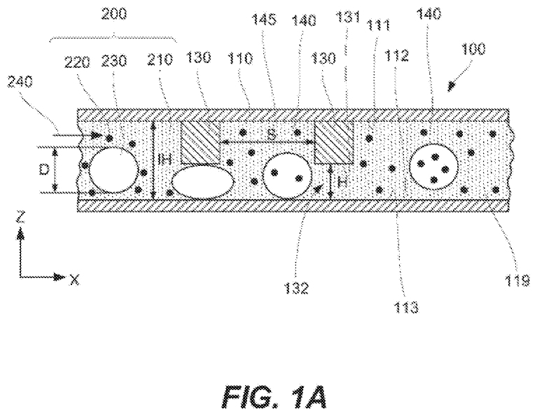

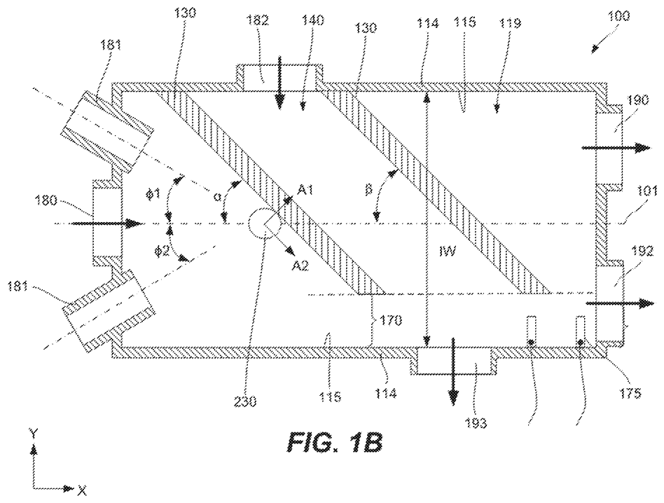

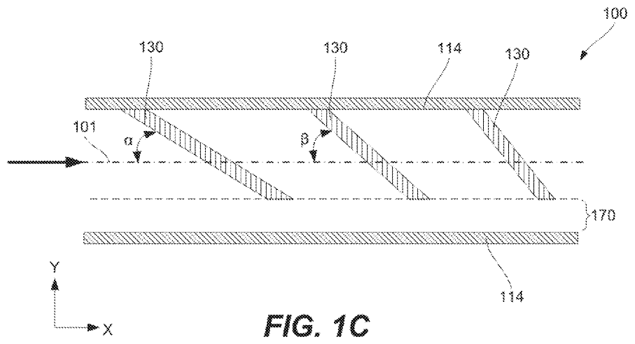

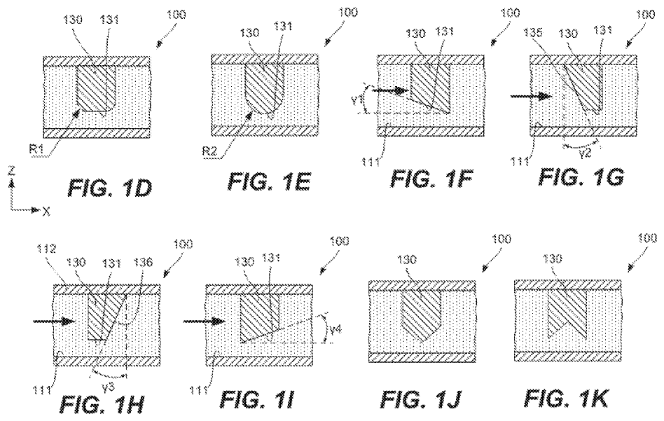

FIG. 1A is a schematic cross-section view of an example cell processing apparatus 100 for intracellular delivery, cell sorting, and/or other operations further described below. In some examples, cell processing apparatus 100 comprises first wall 110 and second wall 112. First wall 110 and second wall 112 may be also referred to as a top wall and a bottom wall, strictly for differentiation and without implying any orientation of cell processing apparatus 100. First wall 110 comprises first interior surface 111. In some examples, first interior surface 111 is planar. However, the interior surface may comprise other shapes. Likewise, second wall 112 comprises second interior surface 113, which may be also planar. In some examples, first interior surface 111 may be parallel to second interior surface 113. First interior surface 111 and second interior surface 113 may extend along the flow direction, identified with arrow 240 in FIG. 1A. First interior surface 111 and second interior surface 113 at least partially define interior 119 of cell processing apparatus 100. More specifically, first interior surface 111 and second interior surface 113 define the interior height (IH), which may impact the linear flowrate within interior 119. Interior 119 may be isolated from the environment and may be used to flow mixture 200, comprising liquid media 210, reagent 220, and cells 230.

In some examples, first wall 110 and/or second wall 112 may be formed from one or more transparent materials. For example, transparent materials of these walls may allow for integration of optical sensors into the cell processing apparatus 100 and/or other types of process control. On the other hand, nontransparent materials for the walls may be used to deliver light-sensitive reagents. Some examples of wall materials may comprise, but not be limited to, polydimethylsiloxane (PDMS), injection molded plastics, silicon, glass, and other polymers.

Referring to FIG. 1A, cell processing apparatus 100 may comprise a plurality of ridges 130, which may extend within interior 119 of cell processing apparatus 100. More specifically, in this example, plurality of ridges 130 may be connected to first wall 110 and extend within from first interior surface 111 and toward second interior surface 113. In some examples, cell processing apparatus 100 may comprises an additional plurality of ridges, which may be connected to the second wall 112 and extend within from second interior surface 113 and toward first interior surface 111. In some cases, the plurality of ridges 130 and the additional plurality of ridges may extend in the opposite direction and, in some examples, they may overlap along the height of the cell processing apparatus 100 (the Z-axis).

FIG. 1A illustrates two ridges forming plurality of ridge 130 extending from first wall 110. However, other numbers of ridges 130 can be used, such as, for example, one ridge, two ridges, three ridges, or four ridges. The number of ridges determines the number of compression cycles that some of cells 230 experience in a single pass through cell processing apparatus 100. Furthermore, additional compression cycles may be achieved by passing cells 230 through cell processing apparatus 100 multiple times. These considerations and the path of cells 230 within cell processing apparatus 100 are further described below.

Each of plurality of ridge 130 may comprises ridge surface 131, forming gap 132 with second interior surface 113. The height (H) of gap 132 may be smaller than the size/diameter (D) of cells 230, which may cause cells 230 to compress as cells 230 pass through gap 132. The compression may also depend on the flowrate and the length of ridge surface 131 (in the X direction), which may be also referred to as a ridge thickness. In some examples, the length of the ridge surface 131 and/or the ridge thickness may be between about 5 micrometers (μm) and 100 micrometers or, between about 20 micrometers and 50 micrometers. The length of the ridge surface 131 may be at least about 1 μm, 2 μm, 3 μm, 4 μm, 5 μm, 6 μm, 7 μm 8 μm, 9 μm, 10 μm, 12 μm, 15 μm, 20 μm, 25 μm, 30 μm, 35 μm, 40 μm, 45 μm, 50 μm, 55 μm, 60 μm, 65 μm, 70 μm, 80 μm, 90 μm, 100 μm, 110 μm, 120 μm, 150 μm, 200 μm, 300 μm, 400 μm, 500 μm, 1 millimeter (mm), or more. In some examples, the length of the ridge surface 131 may be at most about 1 mm, 800 μm, 700 μm, 600 μm, 500 μm, 400 μm, 300 μm, 200 μm, 150 μm, 100 μm, 80 μm, 70 μm, 60 μm, 50 μm, 40 μm, 30 μm, 20 μm, 15 μm, 10 μm, 5 μm, or less.

In some examples, all ridges (or a subset) of the plurality of ridges 130 may have the same length of ridge surface 131 and/or ridge thickness. Alternatively, the length of ridge surface 131 and/or ridge thickness may vary among the ridges. For example, upstream ridges (initial ridges along the flow direction) may have a shorter length of ridge surface 131 than downstream ridges. As such, the compression duration provided by these downstream ridges may be longer than that provided by the upstream ridges. The compression duration may also be impacted by the linear flow rates, which may be controllable by the cross-sectional areas of the cell processing apparatus 100, as further described below.

In some cases, when the length of ridge surface 131 is smaller than the cell size (D), the cell compressions can be compromised due to the cell ability to deform around the ridges, e.g., at least partially remain in uncompressed state when portions of the cell extend outside of gap 132. On the other hand, when the length of ridge surface 131 is much larger than the cell size, such as 10 times or more than the cell diameter, the cells may be prone to accumulation in gaps 132, which can lead to clogging.

Referring to FIG. 1A, in some examples, the cross-sectional profile (in a plane perpendicular to first interior surface 111 and second interior surface 113) of ridge 130 may be rectangular. However, other shapes of the profile are also within the scope, e.g., cylindrical, trapezoidal, or triangular. In some examples, the plurality of compressive surfaces may be orthogonal.

In some examples, ridge surface 131 may be parallel to the second interior surface 113. In other words, gap 132 may be defined by two parallel surfaces, one being ridge surface 131 and another one being a portion of second interior surface 113, and the gap thickness may be constant. Such parallel compressive surfaces may allow for a uniform compression for the entire cell. In some examples, the compression surfaces can be converging and/or diverging. Converging surfaces may allow for increasing the cell compression as the cells pass through the compressive space. Diverging surfaces can be used to allow cell expansion that accelerates cell motion and prevents clogging.

In some examples, the surface roughness of ridge surface 131 may be configured to increase cell membrane poration. For some materials, the surface roughness can be controlled using vapor etching. In some examples, the surface roughness with a mean size of between 10 nanometers (nm) and 1000 nm may be used. In some cases, the surface roughness may have a mean size of at least about 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 20 nm, 50 nm, 100 nm, 300 nm, 500 nm, 800 nm, 1000 nm, 1200 nm, nm, 1300 nm, 1500 nm, or more. In some cases, the surface roughness may have a mean size of less than or equal to about 2000 nm, 1500 nm, 1200 nm, 1000 nm, 800 nm, 600 nm, 500 nm, 400 nm, 300 nm, 200 nm, 100 nm, 50 nm, 20 nm, 10 nm, 5 nm, 1 nm, or less.

In some examples, plurality of ridges 130 may be flexible (e.g., compliant). Flexible ridges may help to reduce cell damage. The ridge flexibility/compliance may be configured by selecting ridge material. In some examples, materials with modulus from 1 to 100 kPa may be used. Furthermore, ridge compliance may be configured using surface coatings with desired elasticity modulus.

Further referring to FIG. 1A, interior 119 may comprise recovery spaces 140, positioned between adjacent pair of plurality of ridge 130 and after the last ridge, along the flow direction/the X direction. In the Z direction, recovery spaces 140 may extend between first wall 110 and second wall 112. The height of recovery spaces 140 (in the Z direction between these walls) may be greater than the gap size. In some examples, the height of the recovery space 140 may be greater than the cell size (D). The height of recovery spaces 140 may be configured to allow the desired cell volume recovery, accompanied by cell expansion in the Z direction. The length of recovery spaces 140 (in the X direction) between two adjacent ridges may be referred to as ridge spacing 145, identified with the letter “S” in FIG. 1A. Ridge spacing 145 may determine the recovery duration, together with the linear flowrate. It has been found that volume gain (V_{gain}) may increase when the recovery time is increased. The recovery time can be increased by increasing ridge spacing 145. Other considerations for determining ridge spacing 145 may comprise cell characteristics, levels of previous compression, and the like. In some examples, ridge spacing 145 may be between 100 micrometers and 1000 micrometers such as between 200 micrometers and 500 micrometers.

Referring to FIG. 1B, cell processing apparatus 100 comprises side walls 114, comprising side interior surfaces 115. Side walls 114 may each be connected to each of first wall 110 and second wall 112, collectively forming interior 119. Side interior surfaces 115 may define the interior width (IW) of cell processing apparatus 100. Together with the interior height (IH), the interior width (IW) may impact the linear flowrate of mixture 200 through interior 119 or, more specifically, through recovery spaces 140. In some examples, the linear flowrate of mixture 200 as it passed through gaps 132 formed by plurality of ridge 130 may be much higher because of a much lower cross-sectional area corresponding to gaps 132 vs. recovery spaces 140 (the volumetric flowrate being the same).

Referring to FIG. 1B, cell processing apparatus 100 may comprise inlet 180 and outlet 190. In some examples, cell processing apparatus 100 may comprises one or more additional inlets 181. For example, multiple inlets may be used for supplying different cells and/or different reagents into cell processing apparatus 100. The inlets may be positioned at various angles relative to the flow direction, which in this example coincides with principal axis 101 of cell processing apparatus 100. For example, inlet 180 is shown to be parallel to the flow direction/principal axis 101. Additional inlets 181 are shown to be not parallel to the flow direction/principal axis 101 (e.g., ϕ1>0° and ϕ2>0°). The angle ϕ1 and/or ϕ2) may be between 20° and 80° or, more in some examples, between 30° and 60°. In some examples, the angle ϕ1 and/or ϕ2) may be greater than or equal to about 5°, 6°, 7°, 8°, 9°, 10°, 12°, 15°, 20°, 25°, 30°, 35°, 40°, 50°, 55°, 60°, 65°, 70°, 75°, 80°, 85°, 90°, or more.

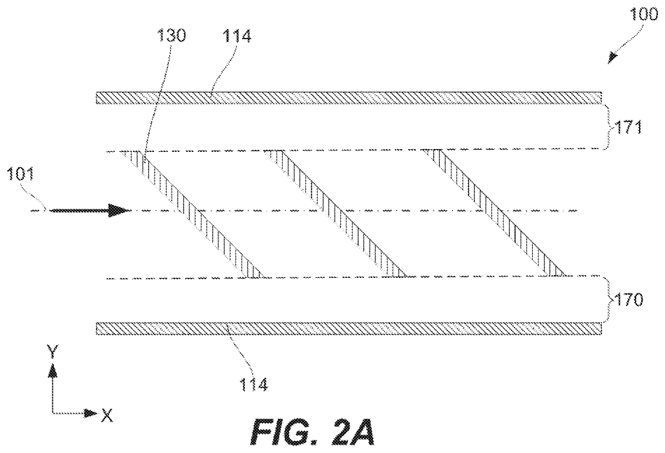

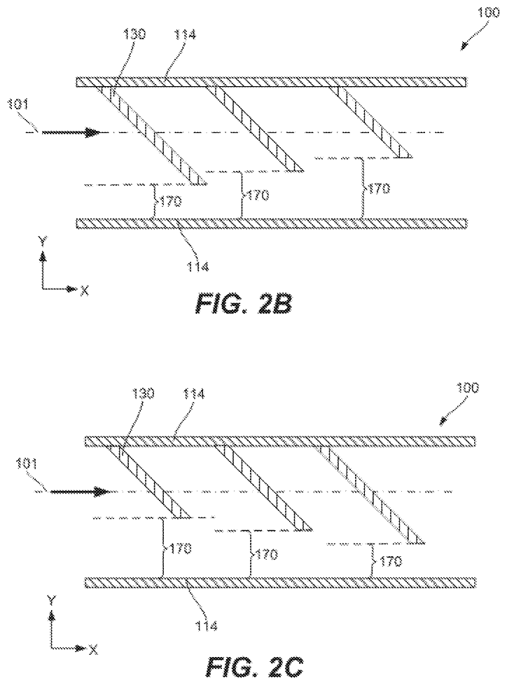

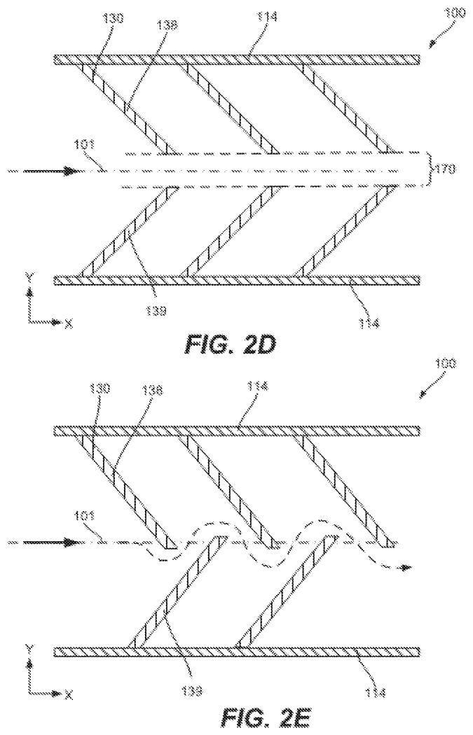

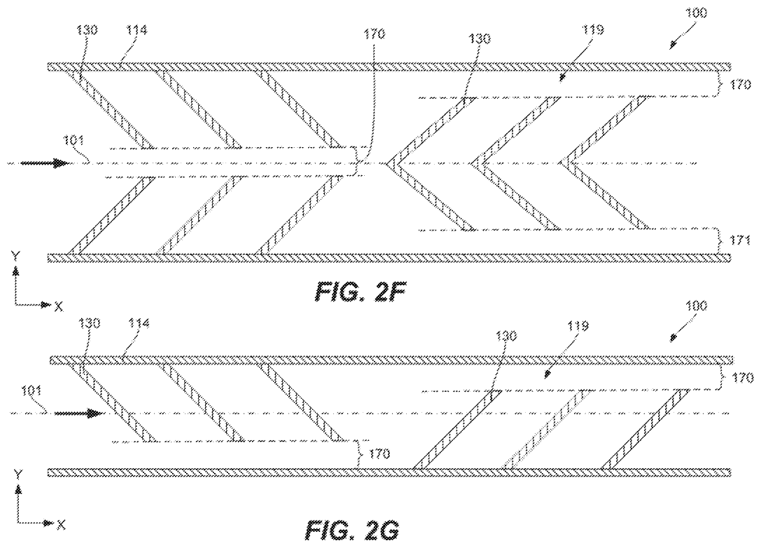

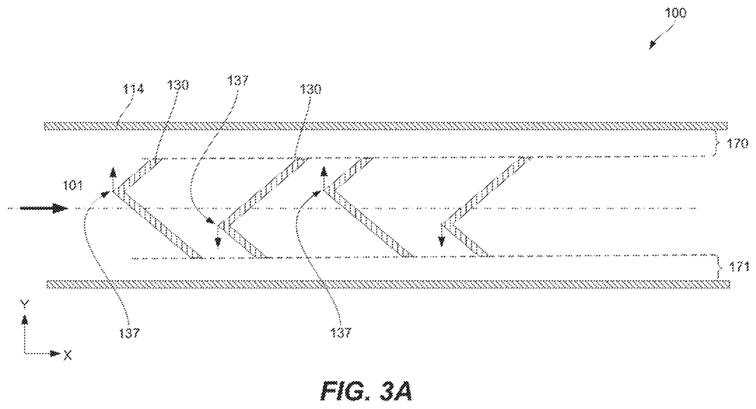

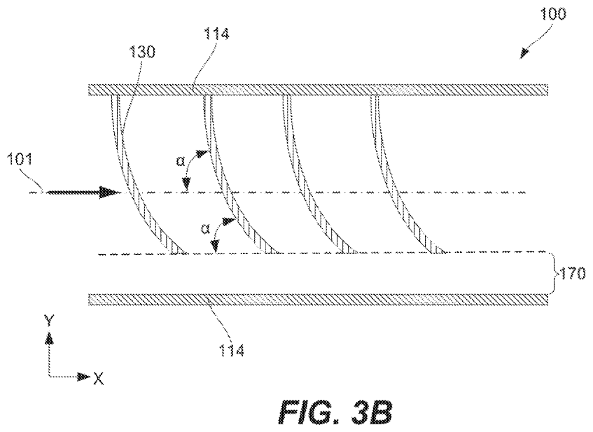

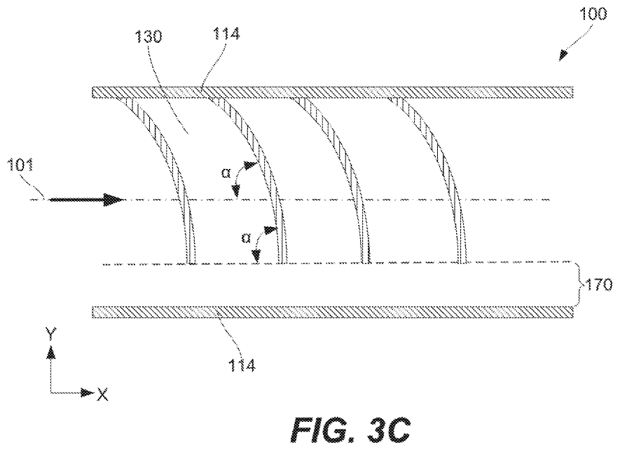

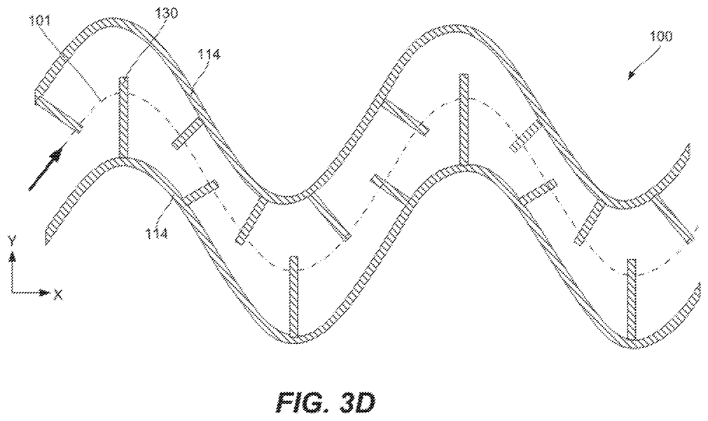

In some examples, inlet 180 may be a self-focusing inlet (e.g. with no sheath focus). The self-focusing inlet may use hydrodynamic focusing, such as Dean's flow effect. For example, inlet 180 may incorporate a focusing section, such as a serpentine channel, focusing ridges, focusing posts, focusing flow splitters, curved geometry using Dean's flow effect, inertial migration effect, and other methods leading to cross-stream cell migration. The focusing section may concentrate cells 230 at desired transverse location within the cell processing apparatus 100. Among other factors, the focusing location depends on the geometry of ridges 130 and ridge surface 131, which may be also referred to as compressive surfaces. For chevron ridges (e.g., shown in FIGS. 2D-2F and FIG. 3A), the focusing location may be at the middle of the channel, in some examples. For diagonal ridges (e.g., shown in FIG. 1B and FIGS. 2A-2C), the focusing location may be biased to the side of diversion channel 170. Without a focusing section, a portion of cells 230 may be able flow from inlet 180 right into diversion channel 170, without being compressed by ridges 130, resulting in nonhomogeneous cell processing. In addition to focusing inlets, hydrodynamic flow may be directed by orientation of ridges 130 as further described below. Furthermore, in some examples, the hydrodynamic flow may be directed using electrical fields, such as electroosmotic flow, electrophoretic flow, and the like. Electrical, magnetic, thermal and other fields can be used to concentrate reagents 220 (e.g., macromolecules, nanoparticles) in specific locations within interior 119 of cell processing apparatus 100 to increase intracellular delivery into cells 230 as cells 230 may be compressed by ridges 130. For example, such effects electrophoresis, electroosmosis, thermophoresis, can be used to concentrate reagents near cells. Electrodes producing the fields can be integrated in walls of cell processing apparatus 100 and controlled by an external controller.

In some examples, a single inlet may be used to reduce an amount of reagents 220 that otherwise can be diluted by focusing a sheath fluid. At outlet 190, processed and unprocessed cells can be mixed for collection. An additional sorting device and operation can be used to separate unprocessed cells from mixture 200 after mixture 200 exists cell processing apparatus 100.

In some examples, cell processing apparatus 100 may comprise intermediate inlet 182, e.g., to introduce different reagents and reagent combinations for multistage cell processing. For example, intermediate inlet 182 may be used to introduce an additional mixture into recovery spaces 140 between adjacent ones of plurality of ridges 130. The composition of this additional mixture may be different from mixture 200, introduced upstream through inlet 180, which may be also referred to as a primary inlet.

In some examples, multiple outlets (e.g., outlet 190 and additional outlet 192) may be used for collecting different types of cells 230. As noted above, cell processing apparatus 100 may have cell sorting capabilities such that different types of cells 230 may flow into different portions of cell processing apparatus 100. Referring to FIG. 1B, less compressible cells may be directed by ridges 130 into diversion channel 170, while more compressible cells may pass through gaps created by ridges 130 and may stay away from diversion channel 170. Outlet 190 may be positioned away from diversion channel 170 and may be used for collecting cells 230 that have undergone compressions by ridges 130. Additional outlet 192 may be aligned with diversion channel 170 and may be used for collecting cells 230, which may be directed into diversion channel 170 and have not been compressed by desired number of ridges of plurality of ridges 130. In general, cell sorting characteristics, which determine whether cells 230 are directed into diversion channel 170 or undergo the compression include viscoelasticity, stiffness, or elasticity, and/or adhesion. Overall, multiple outlets may help to avoid clogging. Any number of outlets can be used one, two, three, four, or more.

In some examples, cell processing apparatus 100 may comprise intermediate outlet 193 as, for example, shown in FIG. 1B. For example, intermediate outlet 193 may be fluidically coupled to diversion channel 170 and open to diversion channel 170. Furthermore, intermediate outlet 193 may be disposed between a pair of plurality of ridges 130 as shown in FIG. 1B. Intermediate outlet 193 may be aligned with recovery space 140 between the pair of plurality of ridges 130. Intermediate outlet 193 may be used for collecting unwanted and abnormal cells and cell clusters, e.g., to prevent clogging of diversion channel 170 without passing these cells through the entire cell processing apparatus 100. In some examples, intermediate outlet 193 may be used to collect subpopulations of processed cells to improve delivery efficiency and uniformity.

Referring to FIG. 1B, all of plurality of ridges 130 may be diagonally-oriented relative to the general flow direction (shown with an arrow and coinciding with principal axis 101 of cell processing apparatus 100) within cell processing apparatus 100, i.e., from inlet 180 to outlet 190. In some cases, the smallest angle between ridges 130 and principal axis 101 may be an acute angle (α<90°). In some examples, the angle may be selected to provide hydrodynamic circulations in gaps 132 under ridges 130 (e.g., between ridge surface 131 and second interior surface 113). The angle of the ridges 130 can also affect the trajectories of cells 230 as, for example, schematically shown by directions A1 and A2 in FIG. 1B. The angle may depend on the flowrate, cell types, and other like parameters. In some examples, the angle may be between 10° to 80° or, more specifically, between 30° and 60°.

In some examples, all of plurality of ridges 130 may have the same angle relative to principal axis 101 (e.g., α=β, referring to FIG. 1B). In these examples, all ridges extend parallel to each other. Alternatively, some ridges in of plurality of ridges 130 may have different angles relative to principal axis 101 (e.g., α≠β) as, for example, is schematically shown in FIG. 1C. For example, a sharper angle may be used closer to inlet 180 (α<β) for early removal of abnormal cells and cell clusters in a less obstructive manner. A larger angle may be used further down the flow path (downstream) for faster cell compression and improved intracellular delivery. Principal axis 101 may be also referred to as the primary flow axis. It should be noted that while the flow may follow the principal axis 101, localized flow may vary, e.g., uncompressible cells may be diverted by a ridge to diversion channel 170.

Referring to FIG. 1B, in some examples, ridges 130 may be in the form of straight bars, individually arranged in interior 119 of cell processing apparatus 100. In some examples, these straight bars may be arranged or even joined together into a chevron pattern as, for example, is shown in FIG. 3A. In this example, each of plurality of ridges may comprise a first ridge portion and a second ridge portion, having different orientations/positioned at different angles relative to the flow direction. It should be noted that the smallest angle between the flow direction and each of the first ridge portion and the second ridge portion may be the same. Alternatively, the smallest angle between the flow direction and each of the first ridge portion and the second ridge portion may be different. Furthermore, this smallest angle may be variable as further described below with reference to FIGS. 3B and 3C.

The shape of each of plurality of ridge 130 may affect or determine the compression profile of cells 230 as they pass through gap 132, created by the ridge, and are compressed by the ridge. Some examples of different ridge cross-sectional shapes of plurality of ridge 130 are illustrated in FIG. 1D-1K. In some examples, all ridges of cell processing apparatus 100 have the same cross-sectional shape. Alternatively, ridges having different cross-sectional shapes may be used in the same cell processing apparatus 100.

FIG. 1D shows an example of ridge 130 with rounded edges/fillets. In some cases, ridge surface 131 may be formed by two rounded edges, which may be separated by a flat portion. These rounded edges may be used to reduce cell damage when cells 230 are compressed by ridge surface 131. In some examples, the radius (R1) of the rounded edges may be between 1 micrometer (μm) and 5 micrometers. In some examples, the radius (R1) of the rounded edges may be at least about 0.5 μm, 1 μm, 2 μm, 3 μm, 5 μm, 6 μm, 7 μm, 8 μm, 10 μm, or more. In some cases, the radius (R1) of the rounded edges may be at most about 10 μm, 8 μm, 7 μm, 6 μm, 5 μm, 4 μm, 3 μm, 2 μm, 1 μm, 0.5 μm or less.

FIG. 1E shows an example of ridge 130, which may be referred to as a rounded ridge. In this example, the entire ridge surface 131 may be non-planar. Such rounded ridges may be used, for example, to mitigate damage to cells 230 with higher mechanical stiffness by providing more gradual compression. Furthermore, in some examples, rounded ridges may be used to reduce channel clogging. In some examples, the radius (R2) of rounded ridges may be between 10 micrometers (μm) and 1000 micrometers. In some examples, the radius (R2) of rounded ridges may be at least about 5 μm, 8 μm, 10 μm, 12 μm, 15 μm, 20 μm, 25 μm, 40 μm, 50 μm, 60 μm, 70 μm, 80 μm, 100 μm, 300 μm, 500 μm, 600 μm, 800 μm, 1000 μm, 1200 μm, or more. In some examples, the radius (R2) of rounded ridges may be at most about 3000 μm, 2000 μm, 1500 μm, 1000 μm, 800 μm, 700 μm, 600 μm, 500 μm, 200 μm, 100 μm, 50 μm, 30 μm, 20 μm, 15 μm, 10 μm, 8 μm, 5 μm, 4 μm, 3 μm, 2 μm, or less.

FIG. 1F shows an example of ridge 130 with inclined ridge surface 131 such that the compression gap decreases as cells 230 pass through the gap and under the ridge surface 131. In some examples, ridges with inclined ridge surface 131, e.g., as shown in FIG. 1F, may be used to improve processing of heterogeneous cell populations. Using such ridges may allow larger cells to enter the compression gap without being deflected to the diversion channel 170. In some examples, the angle (γ1) of ridge surface 131 with a plane parallel to first interior surface 111 may be between 0° and 45° or, in some cases, between 15° and 30°. In some examples, the angle (γ1) of ridge surface 131 with a plane parallel to first interior surface 111 may be at least about 0°, 5°, 10°, 15°, 20°, 30°, 35°, 40°, 45°, 50°, 55°, 60°, 70°, or more. In some examples, the angle (γ1) of ridge surface 131 with a plane parallel to first interior surface 111 may be at most about 70°, 60°, 50°, 45°, 40°, 35°, 30°, 25°, 20°, 15°, 10°, 8°, 7°, 6°, or less.

FIG. 1G shows another example of ridge 130 with inclined ridge front surface 135. The flow direction may be identified with an arrow. In some examples, such ridges may be used to prevent cell accumulation at ridge front surface 135 which may lead to channel clogging. In some examples, the angle of the angle (γ2) of the ridge front surface with a plane perpendicular to first interior surface 111 may be between 0° and 45° or, in some cases, between 15° and 30°. In some examples, the angle of the angle (γ2) of the ridge front surface with a plane perpendicular to first interior surface 111 may be at least about 0°, 5°, 10°, 15°, 20°, 30°, 35°, 40°, 45°, 50°, 55°, 60°, 70°, or more. In some examples, the angle of the angle (γ2) of the ridge front surface with a plane perpendicular to first interior surface 111 may be at most about 70°, 60°, 50°, 45°, 40°, 35°, 30°, 25°, 20°, 15°, 10°, 8°, 7°, 6° or less.

FIG. 1H shows an example of ridge 130 with inclined back surface 136. In some examples, such ridges may be used to direct cells into a portion of recovery space 140 (followed by ridge 130) toward second wall 112 for increased compression by the following ridge. In some examples, the angle of the angle (γ3) of the ridge back surface 136 relative to the plane perpendicular to first interior surface 111 may be between 0° and 35°. In some examples, the angle of the angle (γ3) of the ridge back surface 136 relative to the plane perpendicular to first interior surface 111 may be at least about 0°, 5°, 10°, 15°, 20°, 30°, 35°, 40°, 45°, 50°, 55°, 60°, 70°, or more. In some examples, the angle of the angle (γ3) of the ridge back surface 136 relative to the plane perpendicular to first interior surface 111 may be at most about 70°, 60°, 50°, 45°, 40°, 35°, 30°, 25°, 20°, 15°, 10°, 8°, 7°, 6°, or less.

FIG. 1I shows an example of ridge 130 with inclined surface 131 such that the compression gap may be decreased as cells 230 travel through the compression gap formed by ridge 130. In some examples, ridges shown in FIG. 1I may be used to reduce device clogging due to cells 230 trapped in the compression gap formed by ridges. The gradual increase of the compression gap may reduce the compressive force experienced by cells 230 and generate resultant force which may push the cells 230 to exit the gap. In some examples, the angle (γ4) of ridge surface 131 relative to the plane parallel to first interior surface 111 may be between 0° and 45° or, in some cases, between 15° and 30°. In some examples, the angle (γ4) of ridge surface 131 relative to the plane parallel to first interior surface 111 may be at least about 0°, 5°, 10°, 15°, 20°, 30°, 35°, 40°, 45°, 50°, 55°, 60°, 70°, or more. In some examples, the angle (γ4) of ridge surface 131 relative to the plane parallel to first interior surface 111 may be at most about 70°, 60°, 50°, 45°, 40°, 35°, 30°, 25°, 20°, 15°, 10°, 8°, 7°, 6°, or less.

FIG. 1J shows an example of ridge 130 that forms a decreasing-increasing compression gap. The order of the gap “decreasing” and “increasing” is established by the flow direction shown with an arrow. In some examples, such ridges may be used to improve processing of heterogenous cells and to reduce channel clogging due to cells trapped under the ridges. FIG. 1K shows an example of a ridge that results in an increasing-decreasing gap. In some examples, such ridges may be used to create intermediate relaxation of cells as they are compressed by ridges. This may lead to improved cell volume change.

Referring to FIG. 3A, the first ridge portion and the second ridge portion form leading edge 137. In some examples, these leading edges 137 may not be aligned, e.g., offset relative to principal axis 101 as shown in FIG. 3A. Alternatively, these leading edges 137 may be aligned as, e.g., is shown in FIG. 2F. In some examples, leading edge 137 may be rounded and may have a radius of about the cell diameter (D), e.g., to reduce cell damage due to cell collisions with leading edge 137. The leading edge 137 may be rounded and may have a radius of smaller than the cell diameter (D). The leading edge 137 may be rounded and may have a radius of larger than the cell diameter (D). The leading edge 137 may have other shapes and/or sizes.

FIGS. 3B and 3C are cross-sectional top views of two examples of cell processing apparatus 100, illustrating curved ridges 130. FIG. 3B illustrates an example in which the smallest angle between ridges 130 and principal axis 101 (identified as a) decreases as ridges 130 extend toward diversion channel 170. In this example, it may be easier for the cells 230 positioned closer to diversion channel 170 to be diverted into diversion channel 170 rather than to go through the gap 132 than for the cells 230 positioned further away from the diversion channel 170. This design may make it easier for lesser uncompressible cells (e.g., dead cells, abnormal cells, cell aggregates) to be diverted into diversion channel 170, thereby reducing the chance and/or extent of potential clogging. This example may be used for mixture 200 with a larger variety of cell characteristics.

FIG. 3C illustrates an example in which the smallest angle between ridges 130 and principal axis 101 (identified as a) increases as ridges 130 extend toward the diversion channel 170. In this example, it may be harder for the cells 230 positioned closer to diversion channel 170 to be diverted into the diversion channel 170 rather than going through gap 132 compared to the cells 230 positioned further away from the diversion channel 170. This example may be used for mixture 200 with more uniform cell characteristics.

Returning to FIG. 1B, plurality of ridges 130 forms diversion channel 170, positioned between each of plurality of ridges 130 and one of side interior surfaces 115. In other examples, shown in FIG. 2D-2F, diversion channel 170 may be positioned between two sets of plurality of ridges, such as first ridge set 138 and second ridge set 139. In either case, diversion channel 170 extends along the flow direction and may be used to carry less compressible cells (e.g., dead cells, abnormal cells, cell aggregates). In other words, diversion channel 170 may provide an alternative flow path within interior 119 of cell processing apparatus 100, one path may be through gaps 132 formed by plurality of ridges 130 and another path may be diversion channel 170. The cells 230 may initially go through one or more gaps 132 and then be diverted into the diversion channel 170 or vice versa.

FIG. 2A illustrates the cell processing apparatus 100 with two diversion channels, i.e., diversion channel 170 and additional diversion channel 171. Diversion channel 170 may extend over one of side walls 114, while additional diversion channel 171 may extend over the other one of side walls 114. In general, the cell processing apparatus 100 may comprise any number of diversion channels, which may be extended at various locations.

In some examples, the diversion channel 170 may have the constant width (in the Y direction) along the flow path as, e.g., is shown in FIGS. 1B and 2A. Alternatively, the diversion channel 170 may have a variable width along the flow path as, e.g., is shown in FIGS. 2B and 2C. FIG. 2B is a cross-sectional top view of the cell processing apparatus 100, illustrating the diversion channel 170 increasing in width along the flow direction. FIG. 2C is a cross-sectional top view of the cell processing apparatus 100, illustrating the diversion channel 170 decreasing in width along the flow direction. This width variability may be used to control the flowrate at each location (e.g., each ridge) along the flow path within the cell processing apparatus 100. For example, a portion of the diversion channel 170 with a smaller width may cause the flowrate to increase at that location in comparison to the portion with a larger width.

FIG. 2D is a cross-sectional top view of one example of cell processing apparatus 100, illustrating straight diversion channel 170, positioned away from side walls 114 and along principal axis 101. FIG. 2E is a cross-sectional top view of another example of a cell processing apparatus 100, illustrating a curved diversion channel 170, which may be positioned away from side walls 114 and along principal axis 101. In both examples, a plurality of ridges 130 may comprise a first ridge set 138 and a second ridge set 139 such that first ridge set 138 may not contact second ridge set 139 (e.g., different from a joined chevron pattern shown in FIG. 3A). The first ridge set 138 may extend to and contact one of the side walls 114, while second ridge set 139 may extend to and contact the other one of side walls 114. Referring to the example in FIG. 2D, the first ridge set 138 and second ridge set 139 may not extend past the principal axis 101. The separation of the first ridge set 138 and the second ridge set 139 from the principal axis 101 may create a diversion channel 170 along principal axis 101. Now, referring to the example in FIG. 2E, first ridge set 138 and second ridge set 139 extend past principal axis 101. However, the first ridge set 138 and the second ridge set 139 may be shifted relative to each other along the principal axis 101, thereby avoiding contact between the first ridge set 138 and the second ridge set 139. This overlap and shifting may create a tortious path around the ends of the first ridge set 138 and the second ridge set 139, forming diversion channel 170 toward the plurality of ridges 130 and the gaps 132, e.g., to increase the production yield and/or processing yield.

FIG. 2F is a cross-sectional top view of another example of cell processing apparatus 100, illustrating diversion channel 170 (on the left) splitting into two diversion channels (on the right) and changing the position within interior 119 of cell processing apparatus 100. The left portion of plurality of ridges 130 may be similar to the plurality of ridges 130 shown in FIG. 2D and described above. The diversion channel 170 may be positioned away from the side walls 114 and along the principal axis 101. The right portion of the plurality of ridges 130 may be similar to the plurality of ridges 130 shown in FIG. 3A. This right portion forms diversion channel 170 along one of side walls 114 and additional diversion channel 171 along the other one of side walls 114. This design may provide additional opportunities for processing by the plurality of ridges 130 on the right for cell 230 that were originally directed into the diversion channel 170 on the left.

FIG. 2G is a cross-sectional top view of yet another example of a cell processing apparatus 100, illustrating diversion channel 170, changing the position from one of side walls 114 to the other of side walls 114 within interior 119 of cell processing apparatus 100, in accordance with some examples. This changing is position may provide additional opportunity for processing by the ridges 130 on the right for cell 230 that were originally directed into the diversion channel 170 on the left.

Ridge designs may be selected to reduce an amount of unprocessed cells and enhances the delivery efficiency and uniformity. In some examples, when stiffer cells or cells with larger size encounter a diagonal ridge, they may experience a force that can displace the cells along the ridge. This can result in the separation of cells by stiffness and size with the larger and stiffer cells being displaced in the diversion channel without sufficient processing by compressive surfaces. In this case, the use of overlapping chevron pattern can reintroduce the rejected cells to the trajectories that follow through compressive spaces for additional processing. This in turn can improve the processing of heterogeneous cell populations. It should be note that ridges 130 may have the side of gap 132 that may vary along the width of the ridge. For example, the gap 132 may gradually increase along the width of ridge 130, e.g., toward the diversion channel 170. In this example, the cells 230 that cannot pass through a smaller gap size (e.g., due to their size) are diverted along the ridge and may be able to eventually pass under the ridge as the gap size increases (e.g., closer to diversion channel 170).

In addition to compression of the cells 230, thereby inducing intracellular delivery of the reagent 220 into the cells 230, the ridges 130 may also produce hydrodynamic mixing within the liquid media 210. This hydrodynamic mixing may be due to the various angles of the ridges 130 relative to the flow directions. The hydrodynamic mixing may redistribute the reagent 220 within the liquid media 210 as the reagent 220 is being consumed by the cells 230 during intracellular delivery.

Referring to FIG. 1A, the gap 132 may be selected based on the cell size, the compression needed or desired, and other characteristics of intracellular delivery. In some examples, the gap height (H) may be between 1 micrometers (μm) and 20 micrometers or, in some cases, between 3 micrometers and 8 micrometers. In some examples, the gap height (H) may be at least about 0.5 μm, 1 μm, 5 μm, 10 μm, 15 μm, 20 μm, 30 μm, 40 μm, 50 μm, 60 μm, 70 μm, 80 μm, 90 μm, 100 μm, or more. In some examples, the gap height (H) may be at most about 100 μm, 80 μm, 60 μm, 50 μm, 40 μm, 30 μm, 20 μm, 15 μm, 10 μm, 5 μm, 1 μm, 0.5 μm, or less.

Furthermore, the gap height (H) may be also defined relative to the cell size (D), which may be defined as an average largest cross-sectional dimension of the cells 230. The ratio of the gap height (H) relative to the cell size (D), i.e., H/D, may define the compression level of the cells 230 as they pass through the gap. In some examples, this H/D ratio may be between 15% and 75%, or between 30% and 60%. In some cases, the ratio of the gap height (H) relative to the cell size (D), i.e., H/D, may be at least about 5%, 8%, 10%, 15%, 20%, 30%, 35%, 40%, 50%, 60%, 70%, 80%, 90%, or more. In some cases, the ratio of the gap height (H) relative to the cell size (D), i.e., H/D, may be at most about 99%, 90%, 80%, 75%, 70%, 60%, 65%, 60%, 50%, 30%, 20%, 15%, 10%, or less. Furthermore, in some examples, the cell size (D) may be between 4 micrometers and 20 micrometers or, or in some examples, between 6 micrometers and 15 micrometers. In some cases, the cell size (D) may be at least about 1 μm, 5 μm, 6 μm, 7 μm, 8 μm, 9 μm, 10 μm, 12 μm, 15 μm, 20 μm, 30 μm, 40 μm, 50 μm, 60 μm, 70 μm, 80 μm, 90 μm, 100 μm, or more. In some cases, the cell size (D) may be at most about 100 μm, 80 μm, 60 μm, 40 μm, 20 μm, 10 μm, or less.

FIG. 3D is a cross-sectional top view of the cell processing apparatus 100, illustrating curved side walls 114, in accordance with some examples. In these examples, the curvature may generate hydrodynamic flow circulation within interior 119 of cell processing apparatus 100. The flow circulations emerge due to the Dean effect, leading to Dean vortices. These flow circulations may increase mixing of the cells 230 and the reagents 220, thereby improving the uniformity of intracellular delivery by the compressive ridges 130.

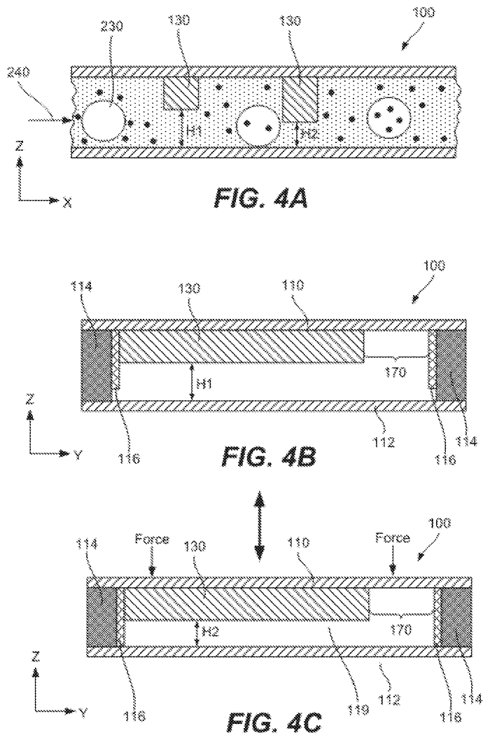

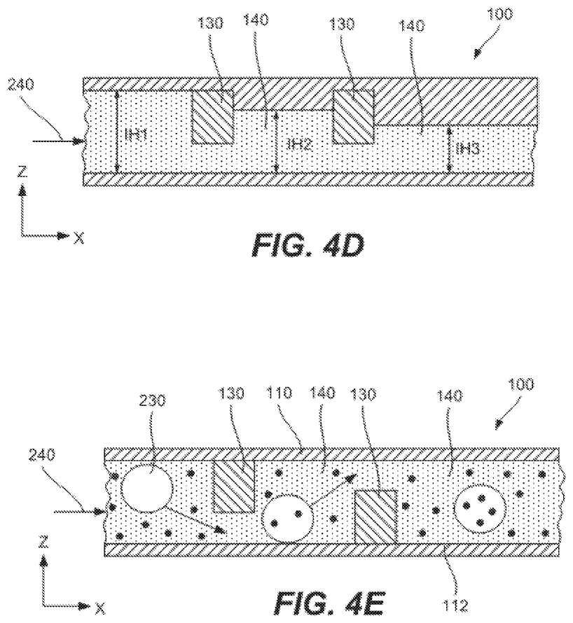

While FIG. 1A illustrates both ridges 130 having the same gap height, other examples are also within the scope. In some examples, the ridges 130 may not have the same gap height. FIG. 4A illustrates an example where ridge 130 on the left has a lager gap height (H1) than a gap height (H2) of ridge 130 on the right. In some example, the gap height decreases along the direction of the flow thereby subjecting cells 230 to higher compression as cells 230 flow through cell processing apparatus 100. With a diverse cell population, a larger gap height earlier in the cell processing apparatus 100 may allow for the compression of larger cells without immediately removing these cells into the side channel 170. Thus, the shorter ridges can be configured for processing larger cells. In some cases, smaller cells may be processed later. When larger cells are compressed in larger gaps, these cells may retain a flattened (pancake-like) shape and can then pass through smaller gaps, in some examples without being removed from the flow. This feature may be referred to as a staged-compression. Furthermore, the smaller gaps may start processing smaller cells, if present (e.g., in a diverse population). In some cases, by varying the gap size, along the channel, the convective intracellular delivery to heterogeneous cell populations may be improved. Furthermore, such channels with varying compression gaps can be used to improve cell sorting by decreasing cell size heterogeneity.

FIGS. 4B and 4C illustrate an example where the first wall 110 and the second wall 112 are movable with respect to each other. For example, the side walls 114 may be flexible. Flexible side walls may allow the first wall 110 and the second wall 112 to be movable with respect to each other. This feature may be used to controllably adjust the gap height in the cell processing apparatus 100. FIG. 4B illustrates a first processing stage, when the first wall 110 and the second wall 112 are positioned further away from each other compared to a second processing stage which is shown in FIG. 4C. The transition from the first processing stage to the second processing stage may be achieved by applying a force between the first wall 110 and the second wall 112 and the compressing side walls 114. In some examples, interior 119 of the cell processing apparatus 100 may comprise spacers 116, which may determine the minimum gap between the first wall 110 and the second wall 112. In other words, the gap height (H2) in the second processing stage may be controlled by the spacers 116, which may be disposed within the interior 119 of the cell processing apparatus 100. Once the force is released, the side walls 114 (e.g., due to their flexibility and resilience) may push the first wall 110 and the second wall 112 back into its original position. This transition may be used for controlling the gap height between the ridge 130 and the second wall 112. For example, the gap height (H1) in the first processing stage may be greater than the gap height (H2) in the second processing stage (i.e., H1>H2).

In some examples, this gap height adjustment may be performed dynamically while flowing liquid media 210 through the cell processing apparatus 100 and even while performing intracellular delivery. For example, the cell processing apparatus 100 may be brought to the second processing stage shown in FIG. 2C for intracellular delivery and, in some cases, back to the first processing stage shown in FIG. 4B for cleaning/flushing. Furthermore, the switch between different processing stages may be performed based on instructions from a system controller as further described below.

In some examples, the interior height (IH) of recovery spaces 140 may change along the length of the cell processing apparatus 100/X-axis/flow direction 240 as, e.g., is shown in FIG. 4D. The interior height (IH) together with the width of each recovery space may determine the linear velocity/linear flow rate of the mixture 200 (comprising media 210 transporting cells 230) within this recovery space. The linear velocity, in turn, may determines the recovery duration and the compression rate of the cells 230. In some examples, the internal height (IH) may be reduced for the recovery spaces 140 as shown in FIG. 4D (IH1>IH2>IH3), e.g., to increase the compression rate of the cells 230 as the cells 230 may be compressed by consecutive ridges. This approach may be used, e.g., to enhance the volume change and may result in an increase of the rate and/or efficiency of intracellular delivery. Alternatively, the internal height (IH) may be increased for the recovery spaces 140. In some examples, the interior height (IH) may change gradually between consecutive ridges to provide gradual changes to the velocity and/or flow rate of the cells.

In some examples, the ridges 130 may be attached to both opposite walls (e.g., first wall 110 and second wall 112) as illustrated in FIG. 4E. Using ridges 130 attached to first wall 110 and the second wall 112, the trajectory and spatial orientation (e.g., between first wall 110 and second wall 112 or along the Z axis) of cells the cells 230 may be altered, as the cells 230 move between consecutive ridges 130 in the recovery spaces 140. In some examples, the ridges attached to the opposite channel walls may be used to induce cell rotation as the cells move through the recovery spaces 140. In some cases, compressed cells (e.g., immediately after leaving from the compression gaps formed by ridges 130) may be characterized by a pancake shape. The cell rotation with recovery spaces 140 may lead to different types of compression (e.g., along a different cell axis) as compared to the previously described compression. In some examples, such variable compressions may be more efficient to cause volume change and intracellular delivery.

The gap height may determine the level of cell deformation that may be needed to pass through the gap. Furthermore, the gap height may determine the flowrate through the gap. For example, when two consecutive ridges in the same cell processing apparatus 100 have different gap heights, the flowrate through the gap having a smaller gap height may be greater.

Intracellular delivery may be controlled and/or adjusted according to the cell compression rate, which may be a rate of volume loss by example cells as they pass through the gap formed by a ridge. The cell compression rate can be determined by flowrate, ridge geometry, a ratio of the gap height to the cell size, the ridge width, ridge angle, and compressive surface coating. Furthermore, the volume loss (Vₗₒₛₛ) may increase with the increase in the cell compression rate. Various processing and device characteristics may be specifically selected to achieve desired cell compression rates.

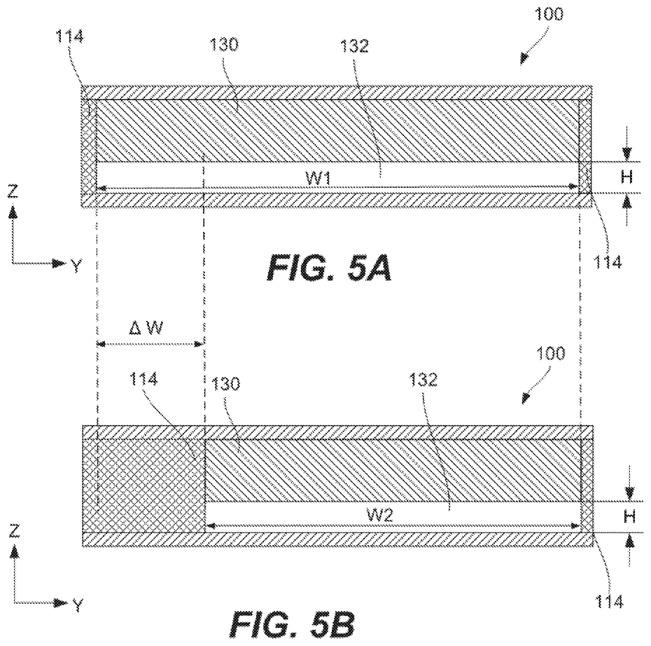

In some example, gaps 132 formed by two ridges 130 in the same cell processing apparatus 100 have different widths as, for example, schematically shown in FIGS. 5A and 5B. In some cases, the width (W1—in the Y direction) of gap 132 in FIG. 5A may be greater than the width (W2—in the Y direction) of gap 132 in FIG. 5B. Because of this gap width difference, the flowrate through gap 132 in FIG. 5A may be slower than through the gap 132 in FIG. 5B even though the gap height (H—in the Z direction) may be the same. As such, the gap width may be used as an additional flow control element, together or instead of the gap height describe below.

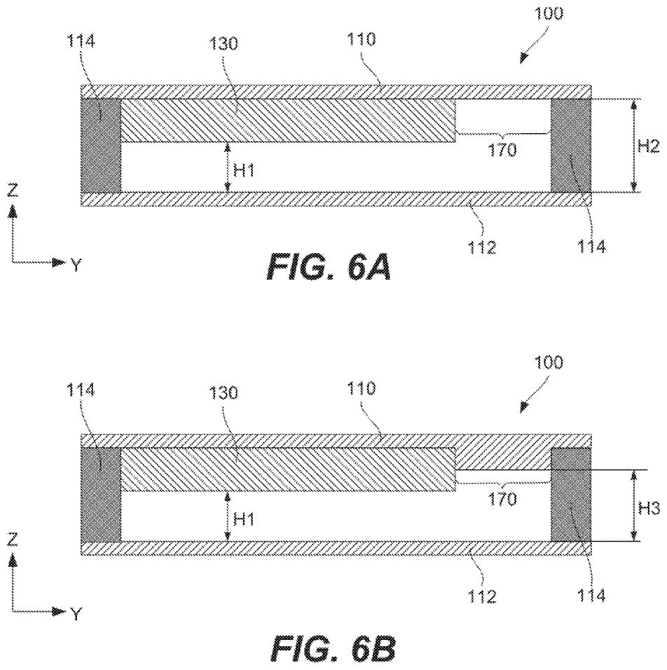

Another flow control may be achieved by varying the cross-sectional area of diversion channel 170. The cross-sectional area may depend on the width and height of diversion channel 170. Varying the width of diversion channel 170 is described above with reference to FIGS. 2B and 2C. Varying the height of diversion channel 170 is described with reference to FIGS. 6A and 6B. In some cases, the height of diversion channel 170 in FIG. 6A (identified as H2) may be greater that the height of diversion channel 170 in FIG. 6B (identified as H3). These heights should be distinguished from the gap height formed by plurality of ridges 130 (and identified as H1 in both figures). When FIGS. 6A and 6B illustrates different portions along the flow direction within the same cell processing apparatus 100, the linear flowrate through the cross-section in FIG. 6A may be less than the flowrate through the cross-section in FIG. 6B. In some cases, the volumetric flowrate may remain constant through any cross-section in cell processing apparatus 100. In some examples, if one cross-sectional area is smaller than the other, the linear flowrate through the smaller cross-sectional area may be greater. The linear flowrate may affect or determine the duration for which the cells 230 may be compressed as these cells pass through the gaps 132 formed by plurality of ridges 130. The cross-sectional area may be a function of the width and the height of the corresponding gap formed by the ridge, in addition to the width and the height of diversion channel 170. The linear flowrate through the recovery spaces 140 may be slower than through the gaps 132, especially if the cross-sectional area of recover spaces 140 is greater than that of the gap 132 and the diversion channel 170.

### Examples of Cell Processing Systems

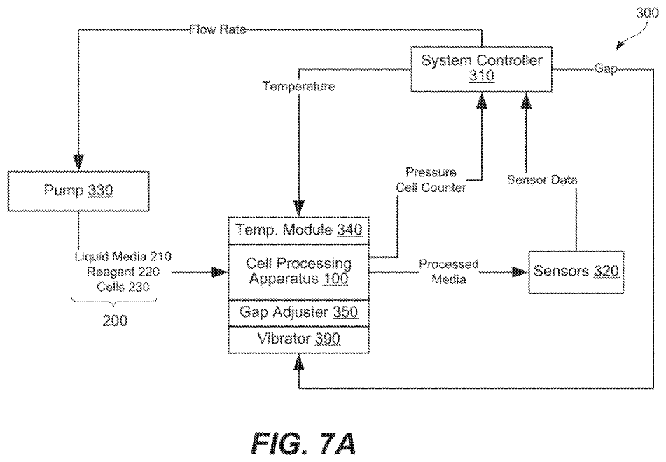

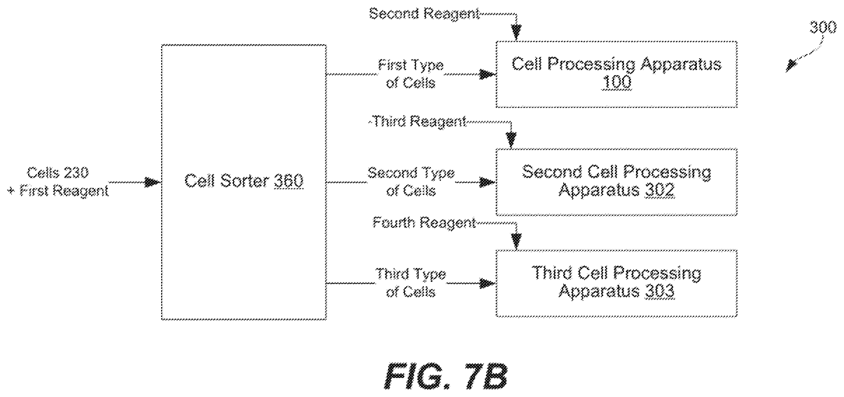

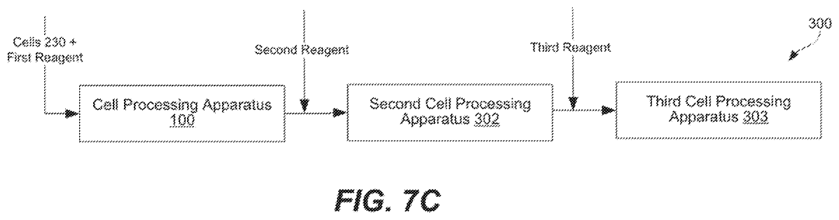

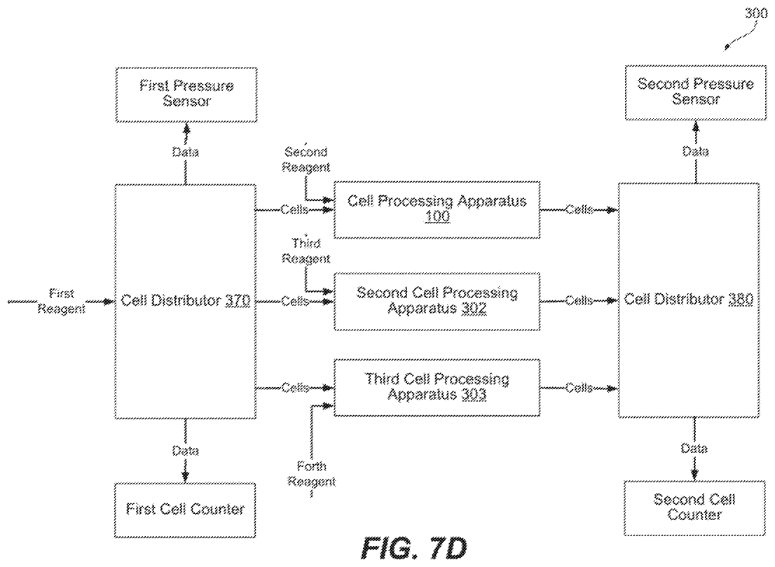

FIG. 7A is a schematic illustration of system 300, comprising a cell processing apparatus 100. Various examples of cell processing apparatuses 100 are described above or elsewhere herein. In some examples, the system 300 may further comprise a system controller 310, one or more sensor(s) 320, and one or more pressure sources (e.g., pump(s) or compressor(s)) 330. The pressure source may generate a positive or negative pressure. The system may comprise any number of pressure sources (e.g., greater than or equal to about 1, 2, 3, 4, 5, 6, 7, 8, 9, 10, 12, 14, 16, 18, 20, or more). The sensors 320 may be configured to measure one or more characteristics of the cells 230, the liquid media 210, and/or the reagents 220 during and after processing, e.g., as the liquid media 210 carrying cells 230 leaves the cell processing apparatus 100. In some examples, the sensors 320 may be integrated into the cell processing apparatus 100 to measure given characteristics within the interior 119 of the cell processing apparatus 100. Some examples of sensors 320 may comprise a temperature sensor (e.g., thermocouple), a cell counter (e.g., Coulter counter, optical counter), a pressure sensor, a flow sensor, a flow control, check valves, control valves, a thermostat, a temperature control chamber, a light measurement unit, a black box, and other sensors, control systems, measurement and/or monitoring tools, or any combination thereof. For example, FIG. 1B illustrates a counter electrode 175 positioned in the diversion channel 170 of the cell processing apparatus 100. Furthermore, cell counters may be positioned at inlet 180 and/or outlet 190 of cell processing apparatus 100, e.g., to control the amount of cells that undergo delivery. Alternatively or additionally, cell counters may be positioned anywhere in the system. The information about the number of cells that is processed can be used to control the quality of the delivery process and to adjust the process parameters. For example, fast reduction in cell count at the outlet compared to the inlet can indicate device malfunction such as clogging and leakage. In combination with reduced pressure, reduced cell count can indicate device leakage, whereas when pressure remains constant or elevated, it can indicate clogging. In the latter case, a cleaning procedure can be started by for example a temporal increase of the flowrate in one or more channels. Furthermore, deviation in these and other controlled parameters can be used to interrupt the delivery procedure, thereby preventing the reduction in the product quality due to the introduction of unprocessed and/or under-processed cells. In some examples, pressure and/or flow sensors may be used to control the flow conditions in one or more channels. In some examples, temperature sensors may be used to control the thermal conditions in the channel.

One or more pumps 330 may be configured to deliver a mixture 200 comprising cells 230, liquid media 210, and/or reagents 220 to the cell processing apparatus 100. The pumps may be fluidically coupled to the inlet 180 of the cell processing apparatus 100. The one or more pumps 330 may control the flowrate, pressure, and other characteristics of the fluid flow, e.g., according to an input provided by the system controller 310.

The system controller 310 may be configured to receive various inputs and/or to control various operations of different components of the system 300. For example, the system controller 310 may receive various sensor data. The system controller 310 may instruct the one or more pumps 330 to increase or decrease one or more flowrates. In some examples, the system controller 310 may instruct the cell processing apparatus 100 to adjust the gaps 132 formed by the compressive elements (e.g., ridges 130).

Optionally, a temperature controlling module 340 may be provided. The temperature controlling module may be thermally coupled to or integrated into the cell processing apparatus 100. For example, the temperature controlling module 340 may be thermally coupled to at least one of the first wall 110 or the second wall 112 of the cell processing apparatus 100 and, in some cases, may be communicatively or operably coupled to the system controller 310. The temperature controlling module 340 may be used to maintain a target temperature. Maintaining a set temperature may improve cell viability and increase the delivery efficiency. Desired temperatures may vary for different processes and/or applications.

The method and systems may further comprise providing a gap adjuster 350, which may be configured to controllably apply a force between the first wall 110 and the second wall 112, which may change the distance between these walls, thereby also changing the gaps formed by the ridges 130 (or other compressive elements). Furthermore, flow regulators, flow sensors, and/or valves may be used for controlling flow conditions. In some examples, actuators may be used to redirect processed and/or unprocessed cells, change flow parameters in the channel(s), and/or induce fluid mixing in the channel to improve delivery.

In some examples, the system 300 may comprise one or more vibrator(s) 390, which may be mechanically coupled to or integrated into the cell processing apparatus 100. Some examples of vibrators 390 may comprise an electromagnetic vibrator, a piezoelectric vibrator, a magnetic vibrator, and a mechanical vibrator. The one or more vibrators 390 may be configured to produce intermittent or continuous vibrations, e.g., at an amplitude of greater than 1 micrometer (μm) and/or at a frequency of greater than 1 Hertz (Hz). The amplitude of the vibrations may be at least about 0.5 micrometers (μm), 1 μm, 5 μm, 10 μm, 15 μm, 20 μm, 30 μm, 40 μm, 50 μm, 60 μm, 70 μm, 80 μm, 90 μm, 100 μm, or more. In some cases, the amplitude of the vibrations may be at most about 200 μm, 150 μm, 100 μm, 80 μm, 60 μm, 40 μm, 20 μm, 10 μm, 5 μm, 1 μm, or less. In some cases, the frequency of the vibrations may be greater than about 0.5 Hz, 1 Hz, 2 Hz, 3 Hz, 5 Hz, 10 Hz, 20 Hz, or more, In some cases, the frequency of the vibrations may be at most about 100 Hz, 80 Hz, 50 Hz, 20 Hz, 10 Hz, 5 Hz, 2 Hz, 1 Hz, 0.5 Hz, or less. In some examples, the one or more vibrators 390 may be communicatively or operably coupled to and/or controlled by the system controller 310, e.g., based on an input from one or more sensors 320.

In some examples, the system 300 may comprise multiple cell processing apparatuses connected in series, in parallel, or a combination thereof, and/or other configurations to each other as, for example, is schematically shown in FIGS. 7B-7D. For example, FIG. 7B illustrates the system 300 comprising a cell sorter 360, connected to three cell processing apparatuses, such as cell processing apparatus 100, the second cell processing apparatus 302, and the third cell processing apparatus 303. The cell sorter 360 may divide the initial set of cells 230 into the first type of cells (delivered to the cell processing apparatus 100), the second type of cells (delivered to the second cell processing apparatus 302), and the third type of cells (delivered to third cell processing apparatus 303). In some examples, the parallel connection may be used to increase cell throughput. For example, the width (in the Y direction) of each cell processing apparatus 100 may be limited by materials and/or operating pressure in order to maintain desired gaps formed by ridges 130. Furthermore, wider cell processing apparatuses may be prone for clogging by abnormal cells and cell clusters. Instead, using multiple parallel cell processing apparatuses, e.g., with a common inlet and outlet, may allow for increasing the throughput without encountering the issues listed above. The inlet and outlet manifolds may be designed to provide equal distribution of flow to all cell processing apparatuses. To ensure that blockage of one cell processing apparatus may not lead to increased flowrates through other cell processing apparatuses, various sensors, e.g., cell counters and pressure transducers, may be integrated into each cell processing apparatus.

FIG. 7C illustrates an example system 300 comprising three cell processing apparatuses connected in series, such that the second cell processing apparatus 302 is positioned between and connected to both cell processing apparatus 100 and the third cell processing apparatus 303. For examples, the cells 230 and the first reagent may be introduced to the cell processing apparatus 100. The processed mixture may be combined with the second reagent prior to introducing into the second cell processing apparatus 302 and then also combined with the third reagent prior to introducing into the third cell processing apparatus 303.

FIG. 7D illustrates the system 300 comprising three cell processing apparatuses connected in parallel and may be referred to as a multiplexed system. The cell distributer 370 may be used to separate the cells 230 into subpopulations that are processed independently by the cell processing apparatus 100, second, the cell processing apparatus 302, and the third cell processing apparatus 303. Each apparatus may be used to deliver different reagents 220 into the cells 230. Processed cells may be then collected in the cell distributor 380. Several systems 300 illustrated in FIG. 7D may be connected in series for delivering additional reagents 220 to one or more cell subpopulation(s).

Multiplexed devices may be used to deliver multiple types of molecules independently to different cells, either in serially connected single channels, or in parallel connected multiple channels with unique molecule inputs to each channel, or in their combination. Multiplexed devices can be used to produce mixtures of cells with different delivered reagents and different combinations of delivered reagents. This can be used to perform multistep delivery into cells. Inline (e.g., series) and/or in parallel, other configurations, and/or combinations thereof of devices can be used to achieve multiplexed delivery.

For example, drug discovery may be accelerated using multiplexed devices where cells may be initially separated into subpopulations that may be used to deliver different reagents to rapidly screen the effects of these reagents on cells to identify reagents which may the most significant efficient on cell functions, or to answer other research questions, or accomplish other goals. Multiple cell sample inlets may be used to process different cells to examine the effect of reagent or reagent-combination on cell function. Furthermore, cells may receive different combinations of reagents. In this case, the device may include several stages of processing where cells may be sequentially delivered different combinations of reagents or different amounts of reagents to evaluate their effects on cell function. To achieve such device functionality, multilayered devices may be used, where different layers of the device(s) may be utilized to perform delivery of specific set of reagents. The processing channels between layers may be connected using in-plane and/or out-of-plane connectors.

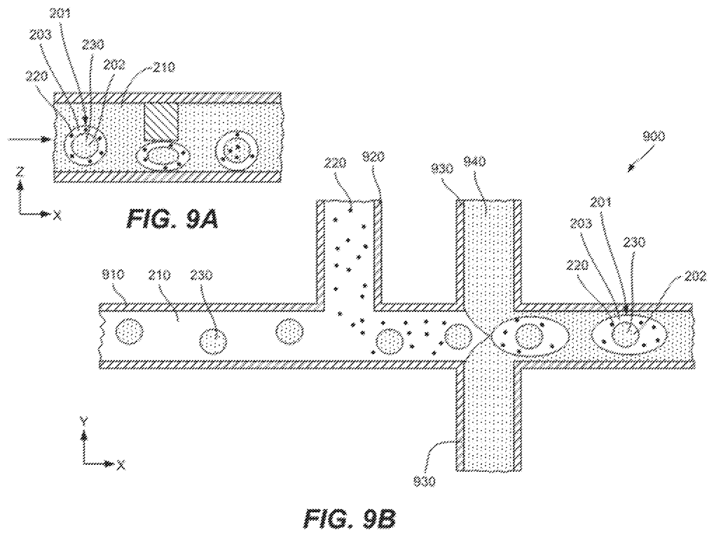

In some examples, a two-phase droplet generator may be positioned before the inlet 180, as described below with reference to FIGS. 9A-9B. One example of such generator comprises a through-oi-flow junction. This generator may be used, for example, to encapsulate cells and deliver reagents, to improve the local concentrations of reagent 220 near the cell 230 as further described herein. In other examples, different types of droplet generators, comprising a flow-focusing junction, a T-junction, or other types may be positioned at various locations in the system for various purposes.

### Examples of Cell Processing Methods

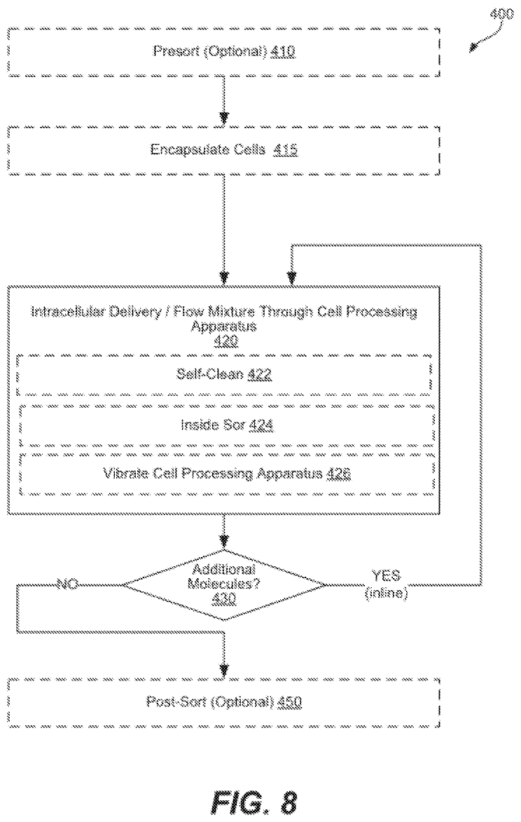

FIG. 8 shows a process flowchart corresponding to method 400 for processing cells 230, in accordance with some embodiments. In some examples, the method 400 involves presorting (block 410). The presorting operation may be used to separate cells into subpopulations based on various characteristics, such as size, mechanical properties (e.g., compressibility), adhesive properties, and the like. Some of these subpopulations (e.g., dead cells, abnormal cells, cell aggregates) may be removed prior to subjecting the remaining cells to intracellular delivery (block 420). Presorting may help reduce the amounts of reagents needed for intracellular delivery, e.g., by adding reagents to the cells with desirable characteristics. Different reagents, different reagent compositions or different amounts of reagents may be used for different cell subpopulations. Furthermore, addition of presorting to the methods and systems may improve uniformity, consistency, and/or more precise control of the intracellular delivery operation thereby yielding higher quality products and maintain a higher cell viability.

In some examples, the presorting operation (block 410) may involve separation using different microfluidic methods such as using gaps formed by diagonal ridges (or other geometries and architectures of ridges and/or microfluidic features) leading to distinct trajectories of cells with different mechanical properties and their separation within the microchannel. In some examples, the separated cells may be then collected at different channel outlets. In some cases, the multiple outlets may be integrated into the system, for example into the microfluidic channels in which intracellular delivery takes place. Presorting may be achieved using microfluidic sorters that may rely on inertial effects leading to equilibration of different cells at different locations within the microfluidic channel, and/or in the flow. Presorting and cell separation may be accomplished using various presorting mechanisms, for example, acoustic streaming effects may discriminate cells with different properties and may lead to different cell positions within the flow. Presorting and separation may be achieved using magnetic forces using magnetic particles attached to the outer membrane of the cells or located inside the cell interior, where the cells may be separated based on the different magnitude of the magnetic force that may be proportional to the amount of the magnetic particles. Cell presorting and separation may be achieved based on cell electrical properties using electrical forces such as using alternating current dielectrophoresis.

In some examples, cell sorting may be used after the intracellular delivery operation and may be referred to post-sorting (block 450). For example, sorting by size and mechanical properties after convective intracellular delivery can be used to remove abnormal cells and nonviable cells from the processed cells. In some examples, post-sorting may be achieved using diagonal ridges that may redirect cells with different properties to follow different trajectories within the cell processing apparatus 100. Other microfluidic and non-microfluidic sorting methods, such as magnetic, acoustic, and electric sorting methods, may be used in other examples. The post-sorting step (block 450), when present, may be used to concentrate the cells 230 and separate them from the reagent 220. In some examples, the separated reagent 220 may be reused for processing additional cells 230 to reduce processing cost.

Intracellular delivery (block 420) may comprise compressing the cells (by passing the cells through the gaps formed by compressive elements (e.g., ridges)) and optionally allowing the cells to recover in between compression stages and after the last compression or at other times or in different sequences. The compressing stage may cause the cells to undergo a loss in intracellular volume (Vₗₒₛₛ). The recovery stages may allow the cells to gain in volume (V_{gain}) and absorb reagents from the surrounding liquid medium. The volume loss and gain may correspond to bulk volume flows across the cell membrane. The volume loss (Vₗₒₛₛ) may depends on the flowrate, gap, cell properties, and any other characteristics. These characteristics may also be interdependent with the compression time. In some examples, the volume loss (Vₗₒₛₛ) during one compression stage may be different than the volume loss (Vₗₒₛₛ) in another compression stage. In some examples, the volume loss (Vₗₒₛₛ) may be at least 10% or 30% of the initial cell volume. The volume loss (Vₗₒₛₛ) depends on the flowrate, gap, cell properties, and other characteristics. These characteristics may be interdependent with the compression time. In some examples, the volume loss (Vₗₒₛₛ) during one compression stage may be different than the volume loss (Vₗₒₛₛ) in another compression stage. In some cases, the volume loss (Vₗₒₛₛ) may be at least about 1%, 5%, 10%, 15%, 20%, 25%, 30%, 35%, 40%, 50%, or more. In some cases, the volume loss (Vₗₒₛₛ) may be at most about 100%, 90%, 80%, 70%, 60%, 50%, 45%, 40%, 30%, 20%, 15%, 10%, or less

In some cases, the cell may gain volume. Volume gain may be achieved after volume loss. For example, a cell may lose some of its volume due to compression by the compressive element and may further gain some or all of its volume back. In some cases, the cell may even gain more volume than its initial volume. The volume gain (V_{gain}) can be characterized in terms of the volume loss (Vₗₒₛₛ). The volume gain (V_{gain}) may depend on the cell properties, recovery time, experimental or operational conditions, flowrate, fluid properties, temperature, pressure, the size and age and type of the cells, the reagent, the device architecture, other features, and other factors. In some examples, the volume loss (Vₗₒₛₛ) during one compression stage may be different from the volume loss (Vₗₒₛₛ) in another compression stage. As an example, volume gain may be at least about 30%, or at least 70% of the volume loss (Vₗₒₛₛ). In some cases, volume gain may be at least about 1%, 5%, 10%, 15%, 20%, 25%, 30%, 35%, 40%, 50%, 80%, 90%, 100%, 110%, 120%, 140%, 150%, 300%, 400% or more of the volume loss. In some examples, volume gain may be at most about 300%, 200%, 100%, 90%, 80%, 70%, 60%, 59%, 30%, 20%, 15%, 10%, or less of the volume loss.

In some examples, intracellular delivery (block 420) may be performed one or more additional times using previously processed cells as schematically shown by the decision block 430 in FIG. 4. In other words, the same set of cells may be passed through multiple cell processing apparatuses arranges in series/in line, in combination and/or in other configurations. Each passage through a cell processing apparatus may be referred to as a separate intracellular delivery stage. Different reagents may be used at each stage.

In some examples, the degree of intracellular delivery may increase by using osmotic effects, leading to cell swelling caused by an increase in the internal cell pressure. Osmotic effects can be controlled using, e.g., the difference of pH levels between the cell interior and media 210.

In some examples, heat shock may be used to temporary alter the state of the cells 230, which can enhance the intracellular delivery. The heat shock may be generated by one or more heating elements integrated into cell processing apparatus 100. In some cases, each heating element may provide localized heating as the cells 230 flow though the cell processing apparatus 100. Heating may be configured such that the heat shock experienced by the cells 230 may not exceed the critical time leading to cell damage. In some examples, the heat shock may be less than 1 second. The heat shock may be less than 30 seconds (s), 25 s, 20 s, 15 s, 10 s, 9 s, 8 s, 7 s, 6 s, 7 s, 5 s, 4 s, 3 s, 2 s, 1.5 s, 0.9 s, 0.8 s, 0.7 s, or less. In other examples, the heat shock may be more than about 0.2 s, 0.3 s, 0.4 s, 0.5 s, 0.6 s, 0.7 s, 0.8 s, 0.9 s, 1 s, 1.5 s, 2 s, 3 s, 4 s, 5 s, 6 s, 7 s, or more.

In some examples, cooling may be used to reduce the temperature of the cells 230 and/or the reagent 220 and/or to maintain a reduced temperature of the cells 230 and the reagent 220. In some examples, the temperature reduction may be configured to suppress adverse cell response to the reagents and/or to increase cell viability. Different methods may be used to achieve temperature control in the channel. For example, incorporating of a Peltier element into the cell processing apparatus 100 may be used to achieve thermoelectric cooling of cells 230 and reagent 220. The temperature may be between 1° C. to 50° C., be between 1° C. to 40° C., be between 1° C. to 35° C., be between 1° C. to 30° C., be between 1° C. to 25° C., be between 1° C. to 20° C., be between 1° C. to 15° C., be between 1° C. to 10° C., be between 3° C. to 30° C., be between 5° C. to 30° C., be between 5° C. to 20° C., or be between 10° C. to 20° C. In some examples, the temperature may be between 5° C. and 30° C. In some cases temperature may be between 10° C. to 20° C.

In some examples, the state of the cells 230 may be altered using light, which in some cases, can enhance the intracellular delivery. In some examples, a light source may be integrated into the cell processing apparatus 100 (e.g., using transparent walls of the cell processing apparatus 100). Light sources with different wavelength may be used to modify the cell state and the cell response to the reagents.

In some examples, electric fields and/or magnetic fields may be used to enhance the transport of the reagents 220 into the interior of cells 230. For example, electrical fields may be generated by electrodes integrated into the walls of the cell processing apparatus 100. Electric fields may induce the motion of charged molecules, e.g., to increase the delivery of reagents into the cell interior. In some examples, permanent magnets and/or electric magnets may be used to generate magnetic fields of desired strengths which may depend on the magnetic properties of the reagents.

In some examples, immune suppressing reagents may be used to suppress the adverse cell response to delivered reagents and/or to improve the incorporation of reagents into the cells 230 and the cell viabilities.

In some examples, intracellular delivery (block 420) may comprise self-cleaning (block 422) of the cell processing apparatus 100. FIG. 1B illustrates a top cross-sectional view of cell processing apparatus 100 showing channel 170, disposed between trailing ends of ridges 130 and side wall 114. One set of cells 230, which may be referred to as a first set, may be able to pass through the gaps formed by ridges 130 as schematically shown by A1 direction in FIG. 1B. Another set of cells 230, which may be referred to as a second set, may not be able to pass through these gaps and may be directed along ridges 130, e.g., along A2 direction shown in FIG. 1B. This second set may comprise various abnormal cells (e.g., cells with low compressibility) or cell clusters. The second set of cells may travel along the ridges 130 until they reach channel 170, which may allow these cells to flow through the rest of cell processing apparatus 100 without encountering any further ridges or other flow restrictions. As such, this second set may be effectively removed from the flow path of the first set, which may prevent clogging and other negative effects during intracellular delivery of the first set. Overall, this self-cleaning feature may allow operating the cell processing apparatus 100 with fewer interruptions and changes to the flow (e.g., reversing the flow).

In some examples, self-cleaning may be achieved using an axillary fluid flow along ridges 130. For example, a temporary reverse flow may be used to separate cells from the ridges 130. Furthermore, other forces, such as electric forces, magnetic forces may be used to transport the cells to the channel 170.

Intercellular delivery (block 420) may be performed at various flowrates such as between 1 millimeter per second (mm/s) and 500 millimeters per second or, in some cases 5 millimeters per second and 200 millimeters per second, where the velocity is the average velocity of the fluid. Flowrates may be at least about 1 mm/s, 2 mm/s, 3 mm/s, 4 mm/s, 5 mm/s, 6 mm/s, 7 mm/s, 8 mm/s, 9 mm/s, 10 mm/s, 15 mm/s, 20 mm/s, 30 mm/s, 40 mm/s, 50 mm/s, 60 mm/s, 70 mm/s, 80 mm/s, 90 mm/s, 100 mm/s, 150 mm/s, 180 mm/s, 200 mm/s, 210 mm/s, 220 mm/s, 230 mm/s, 240 mm/s, 250 mm/s, 300 mm/s, 400 mm/s, 450 mm/s, 500 mm/s, or more. In some examples, flowrates may be at most about 1 mm/s, 2 mm/s, 3 mm/s, 4 mm/s, 5 mm/s, 6 mm/s, 7 mm/s, 8 mm/s, 9 mm/s, 10 mm/s, 15 mm/s, 20 mm/s, 30 mm/s, 40 mm/s, 50 mm/s, 60 mm/s, 70 mm/s, 80 mm/s, 90 mm/s, 100 mm/s, 150 mm/s, 180 mm/s, 200 mm/s, 210 mm/s, 220 mm/s, 230 mm/s, 240 mm/s, 250 mm/s, 300 mm/s, 400 mm/s, 450 mm/s, 500 mm/s. Flowrates may controls and/or affect the cell compression rates. Furthermore, the flowrate may affect and/or controls the pressure at the inlet and within the cell processing apparatus 100.

In some example, a variable flowrate may be used during intercellular delivery (block 420), which may be also referred to as an unsteady flow condition. For example, the flowrate may change at a predefined, or random pattern during the intracellular delivery. For example, pulsating flow may be used such as the flowrate can oscillate with amplitude between 10% and 50% of the average flowrate with the frequency between 0.1 Hz and 100 Hz. The flowrate may oscillate with an amplitude of at least about 5%, 8%, 9%, 10%, 11%, 12%, 13%, 14%, 15%, 16%, 17%, 18%, 19%, 20%, 22%, 25%, 27%, 30%, 35%, 40%, 50%, 52%, 55%, 60%, or more of the average flowrate. In some cases, the flowrate may oscillate with an amplitude of at most about 70%, 60%, 55%, 50%, 45%, 35%, 30%, 25%, 20%, 15%, 10%, 5%, or less of the average flowrate. The oscillation frequency in each case may be at least about 0.05 Hz, 0.1 Hz, 0.2 Hz, 0.5 Hz, 1 Hz, 1.5 Hz, 2 Hz, 3 Hz, 4 Hz, 5 Hz, 6 Hz, 7 Hz, 8 Hz, 9 Hz, 10 Hz, 15 Hz, 20 Hz, 25 Hz, 30 Hz, 40 Hz, 50 Hz, 60 Hz, 70 Hz, 80 Hz, 90 Hz, 95 Hz, 100 Hz, 105 Hz, 110 Hz, 120 Hz, 130 Hz, 140 Hz, 150 Hz, 200 Hz, 300 Hz, or more. In some cases, the flowrate may oscillate with a frequency of at most about 400 Hz, 350 Hz, 300 Hz, 250 Hz, 200 Hz, 150 Hz, 120 Hz, 110 Hz, 100 Hz, 92 Hz, 90 Hz, 85 Hz, 80 Hz, 75 Hz, 70 Hz, 65 Hz, 60 Hz, 50 Hz, 40 Hz, 30 Hz, 25 Hz, 20 Hz, 15 Hz, 10 Hz, 5 Hz, 2 Hz, 1 Hz, 0.9 Hz, 0.8 Hz, 0.7 Hz, 0.6 Hz, 0.5 Hz, 0.4 Hz, 0.3 Hz, 0.2 Hz, 0.1 Hz, 0.09 Hz, 0.08 Hz, 0.07 Hz, 0.06 Hz, 0.05 Hz, or less.

In another example, the flowrate can be reduced to substantially zero and then restored to the about the full flow velocity. Flowrate may change according to other patterns. The variable flowrate may be used to temporary increase the hydrodynamic force applied on the cells as these cells pass through the gaps formed by the ridges 130, e.g., to cause faster cell compressions. Furthermore, in some cases, the unsteady flow may improve the removal of abnormal cells and cell clusters from ridges 130 and into diversion channel 170, e.g., to prevent clogging of the cell processing apparatus 100. For example, the flowrate may increase by a magnitude between 20% and 100% for a period of time between about 0.1 second to 5 second. In another example, the flowrate can be reversed with the magnitude between 20% and 200% of the forward velocity for a period of time from 0.1 second to 5 second. The flowrate may increase or decrease by a magnitude of at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99.99%, or more. The flowrate may increase or decrease by a magnitude of at most about 100%, 99.9%, 99%, 98%, 95%, 90%, 80%, 85%, 80%, 75%, 70%, 65%, 60%, 50%, 45%, 40%, 30%, 25%, 20%, 15%, 10%, 5%, 2%, 1% or less. The change in the flowrate or the direction of fluid flow in each case may be temporary or stable according to the desired conditions. For example, in each of the conditions, the increased or decreased flowrate may last for a time period of at least about 0.05 seconds (s), 0.1 s, 0.15 s, 0.2 s, 0.3 s, 0.4 s, 0.5 s, 0.6 s, 0.7 s, 0.8 s, 0.9 s, 1 s, 2 s, 3 s, 4 s, 5 s, 6 s, 7 s, 8 s, 9 s, 10 s, 15 s, 20 s, 30 s, or more. For example, in each of the conditions, the increased or decreased flowrate may last for a time period of at most about 0.005 seconds (s), 0.05, 0.1 s, 0.15 s, 0.2 s, 0.3 s, 0.4 s, 0.5 s, 0.6 s, 0.7 s, 0.8 s, 0.9 s, 1 s, 2 s, 3 s, 4 s, 5 s, 6 s, 7 s, 8 s, 9 s, 10 s, 15 s, 20 s, 30 s, or less. In some cases, the flowrate increase/decrease may be in the form of a pulse of fluid flow.

In some examples, the intracellular delivery or flowing the mixture through the cell processing apparatus 100 (block 420) may comprise or be combined with inside sorting (block 424), which may occur within the interior of the cell processing apparatus. For example, as the cells with different properties flow through the cell processing apparatus, some cells may not be able to pass through the gaps formed by the ridges, e.g., due to mechanical properties, viscoelastic properties, size, adhesive properties, or other properties or characteristics of the cell, or flow, or other reasons and/or factors. The ridges may direct such cells, or a subset of such cells into a diversion channel as described anywhere herein, for example with reference to FIG. 1B. In some cases, cells with given properties can be sorted out before they undergo molecular delivery. As a result, only selected cell subpopulations may undergo molecular delivery.

In some examples, intracellular delivery/flowing the mixture through the cell processing apparatus 100 (block 420) may comprise vibrating the cell processing apparatus 100 (block 426). As described above with reference to FIG. 7A or elsewhere herein, in some examples, system 300 may comprise one or more vibrators 390, which may be mechanically coupled to or integrated into the cell processing apparatus 100. The vibrations of the cell processing apparatus 100 may be intermittent or continuous. In some examples, intermittent vibrations may last between 0.001 seconds and 600 seconds or, in some cases, between about 0.1 seconds (s) and about 10 seconds. In some cases, intermittent vibrations may last for at least about substantially zero seconds (s), 0.001 s, 0.0015 s, 0.002 s, 0.0025 s, 0.003 s, 0.004 s, 0.005 s, 0.006 s, 0.007 s, 0.008 s, 0.009 s, 0.01 s, 0.02 s, 0.03 s, 0.04 s, 0.05 s, 0.06 s, 0.07 s, 0.08 s, 0.09 s, 0.1 s, 0.5 s, 1 s, 2 s, 5 s, 10 s, 20 s, 30 s, 40 s, 50 s, 60 s, 70 s, 80 s, 90 s, 100 s, 200 s, 300 s, 400 s, 500 s, 600 s, 700 s, 800 s, or more. In some cases, intermittent vibrations may last for at most about 800 s, 700 s, 650 s, 600 s, 500 s, 450 s, 400 s, 350 s, 300 s, 250 s, 200 s, 150 s, 100 s, 90 s, 80 s, 70 s, 60 s, 50 s, 40 s, 30 s, 20 s, 10 s, 5 s, 1 s, 0.5 s, 0.1 s, 0.015 s, 0.01, 0.005 s, 0.001 s, or less.

The vibrations may be initiated or changed in response to the input received from sensors 320. In some examples, the amplitude of the vibrations may be greater than about 1 micrometer (μm). In the same or other examples, the frequency of the vibrations may be greater than 1 Hz. In some examples, the amplitude of vibrations may be at least about 0.001 μm, 0.005 μm, 0.006 μm, 0.007 μm, 0.008 μm, 0.009 μm, 0.01 μm, 0.02 μm, 0.05 μm, 0.08 μm, 0.09 μm, 1 μm, 1.1 μm, 1.5 μm, 1.6 μm, 1.7 μm, 1.8 μm, 1.9 μm, 2 μm, 2.1 μm, 2.2 μm, 2.3 μm, 2.4 μm, 2.5 μm, 3 μm, 5 μm, 10 μm, or more. In some examples, the amplitude of vibrations may be at most about 10 μm, 8 μm, 5 μm, 4 μm, 2 μm, 3 μm, 2 μm, 1.5 μm, 1.2 μm, 1.1 μm, 1 μm, 0.9 μm, 0.5 μm, 0.1 μm, or less. In the same or other examples, the frequency of the vibrations may be at least about 0.05 Hz, 0.1 Hz, 0.2 Hz, 0.5 Hz, 1 Hz, 1.5 Hz, 2 Hz, 3 Hz, 4 Hz, 5 Hz, 6 Hz, 7 Hz, 8 Hz, 9 Hz, 10 Hz, 15 Hz, 20 Hz, 25 Hz, 30 Hz, 40 Hz, 50 Hz, 60 Hz, 70 Hz, 80 Hz, 90 Hz, 95 Hz, 100 Hz, 105 Hz, 110 Hz, 120 Hz, 130 Hz, 140 Hz, 150 Hz, 200 Hz, 300 Hz, or more. In some cases, in the same or other examples, the frequency of the vibrations may be at most about 400 Hz, 350 Hz, 300 Hz, 250 Hz, 200 Hz, 150 Hz, 120 Hz, 110 Hz, 100 Hz, 92 Hz, 90 Hz, 85 Hz, 80 Hz, 75 Hz, 70 Hz, 65 Hz, 60 Hz, 50 Hz, 40 Hz, 30 Hz, 25 Hz, 20 Hz, 15 Hz, 10 Hz, 5 Hz, 2 Hz, 1 Hz, 0.9 Hz, 0.8 Hz, 0.7 Hz, 0.6 Hz, 0.5 Hz, 0.4 Hz, 0.3 Hz, 0.2 Hz, 0.1 Hz, 0.09 Hz, 0.08 Hz, 0.07 Hz, 0.06 Hz, 0.05 Hz, or less.

In some examples, method 400 may further comprise cell encapsulation (block 415). In some cases, increasing the concentration of reagents around cells during intracellular delivery may increase the amount of reagents entering the cells. However, supplying large amounts reagents can be challenging due to higher costs or other factors. In some examples, the cells may be encapsulated into a shell which may have a high concentration of one or more reagents as, for example, as schematically shown in FIG. 9A. As an example, FIG. 9A illustrates a core-shell structure 201 comprising the cells 230 (operable as a core 202) and the shell 203. The core-shell structure 201 may be also referred to as a two-phase system.

The shell 203 may surround the cell 230 and may comprise the reagent 220. The core-shell structure 201 may be suspended in liquid media 210, which in these examples may otherwise be free from reagents (i.e., besides reagent 220 in shell 222). Providing reagent 220 in the shell 203 may allow substantially increasing the concentration of reagent 220 at the surface of shell 203 in comparison to dispersing the same amount of reagent 220 in the entire liquid media 210. In some examples, the shell 203 may be a vesicle or in an oil/water droplet.

FIG. 9B is a cross-sectional side view of a two-phase droplet generator 900, illustrating the formation of an example core-shell structures 201, in accordance with the methods and systems provided herein. The droplet generator may have a first inlet 910, a second inlet 920, a third inlet 930, or more inlets. The first inlet 910 may be used to supply media 210 with the cells 230. Inlet 920 may be used to supply the reagent 220. Inlets 930 may be used to supply a second media 940 or more media streams. In some examples, liquid media 210 and second media 940 may be immiscible, such as water and oil. In other examples, the liquid media 210 and the second media 940 may be miscible. The surface tension between the liquid media 210 and the second media 940 may leads to the formation of droplets. In some examples, the flow rate through each inlet may be independently controlled. For example, the flow rates through the first inlet 910 and the second inlet 920 may be configured to achieve desired concentrations of the reagent 220 near the cells 230. In some examples, the flow rate through second inlet 920 may be slower, substantially slower, slightly slower, or much slower than the flow rate through the first inlet 910. In some examples the flow rate through the second inlet may be at least about 2 times, 2.5 times, 3 times, 3.5 times, 4 times, 4.5 times, 5 times, 5.5 times, 6 times, 7 times, 8 times, 9 times, 11 times, 12 times, 13 times, 14 times, 15 times or to a higher extent slower than the flowrate through the first inlet. In some examples, the flow rate through the second inlet may be at most about 30 times, 20 times, 15 times, 10 times, 8 times, 5 times, 4 times, 3 times, twice, or less slower than the flow rates through the first inlet. In other examples, the flow rate through the first inlet may be at least about 2 times, 2.5 times, 3 times, 3.5 times, 4 times, 4.5 times, 5 times, 5.5 times, 6 times, 7 times, 8 times, 9 times, 11 times, 12 times, 13 times, 14 times, 15 times or more slower than the flowrate through the second inlet. In some examples, the flow rate through the first inlet may be at most about 30 times, 20 times, 15 times, 10 times, 8 times, 5 times, 4 times, 3 times, twice, or less extents slower than the flow rates through the second inlet. The flow rates through the first inlet 910 and the third inlets 930 may be configured to achieve the formation of individual droplets of the desired size. The size of these droplets and the droplet generation frequency may depend on the difference between the flow rates through the first and the second inlet (or the ratios thereof), medium viscosities, surface tension, channel size, channel cross-section geometry, and/or other factors. In some examples, the size of droplets and the droplet generation frequency may be configured to obtain single cell in each droplet. Single cell encapsulation in droplets may follow a Poisson distribution. Poisson distribution may be shifted to improve higher single cell encapsulation frequencies by adjusting variables such the density of the cells in the inlet stream, the droplet size, the flowrates through each of the inlets, fluid properties, the reagents, and other factors. In some cases, droplet sorting techniques may be applied to separate droplets containing single cells from other droplets, such as empty droplets (droplets not containing any cells), and/or droplets containing multiple cells. Droplets may be monodispersed. For example, droplets may comprise substantially similar sizes. Alternatively, droplets may comprise various size. The size of droplets may comprise a distribution. The single cell efficiency in the droplets may depend on droplet sizes. In some cases, it may be desired to have a monodisperse droplet population and/or avoid droplet sizes that may be too small. In some cases, very small droplets may not encapsulate cells, and be left empty.

In some examples, surfactants may be used to configure the formation of droplets. Surfactant may be added to the oil stream. Surfactants may be fluorinated surfactants. In some cases, the addition of the surfactant to the oil may help stabilize the formed droplets.

### Computer Systems

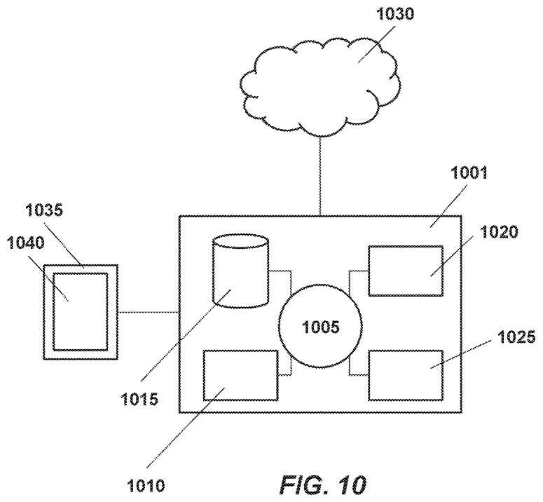

The present disclosure provides computer systems that are programmed to implement methods of the disclosure. FIG. 10 shows a computer system 1001 that is programmed or otherwise configured to perform the methods of the present disclosure. For example, the compute system can be programmed or otherwise configured to control flow pumps, temperature (cooling or heating), supply of reagents, and receive information about flow rate, pressure in the channels, cell count, cell viability, device clogging, channel temperature, as well as conditions of cells, media and reagents before and after the processing. The computer system 1001 can regulate various aspects of the methods of the present disclosure. The computer system 1001 can be an electronic device of a user or a computer system that is remotely located with respect to the electronic device. The electronic device can be a mobile electronic device. The computer system 1001 includes a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. The network 1030, in some cases with the aid of the computer system 1001, can implement a peer-to-peer network, which may enable devices coupled to the computer system 1001 to behave as a client or a server.

The computer system 1001 includes a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and write back.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user (e.g., a scientist or technician). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1001 via the network 1030.

Methods as described herein can be implemented by way of machine (e.g., computer processor) executable code stored on an electronic storage location of the computer system 1001, such as, for example, on the memory 1010 or electronic storage unit 1015. The machine executable or machine-readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display 1035 that comprises a user interface (UI) 1040 for providing, for example, relevant information of intracellular delivery such as parameters of microfluidic devices used for delivery, cell type, molecules to be delivered into the cells, and/or results of the delivery. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1005. The algorithm can, for example, regulate one or more parameters of the methods and/or systems (e.g., flow rate, temperature, pressure, buffer solution, cell type etc.).

In some cases, the computer systems may comprise or be operatively coupled to an imaging system to control cell state, cell deformation, cell volume change, delivery of reagents.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1. A cell processing apparatus comprising:
a first wall comprising a first surface, wherein the first wall extends along a flow direction;
a second wall comprising a second surface, wherein the second wall extends along the flow direction;
a plurality of ridges connected to the first wall, wherein the plurality of ridges extends from the first surface toward the second surface, and wherein a ridge of the plurality of ridges comprises a ridge surface that forms a gap with the second surface; and
a diversion channel extending along the flow direction, wherein:
the diversion channel is at least partially defined by at least a subset of the plurality of ridges, and
a width of the diversion channel, measured perpendicular to the flow direction, is variable along the flow direction.

2-5. (canceled)

6. The cell processing apparatus of claim 1, wherein at least two ridges of the plurality of ridges are oriented at different angles relative to the flow direction.

7-8. (canceled)

9. The cell processing apparatus of claim 1, wherein the plurality of ridges each has a ridge surface that forms a gap with the second wall, and wherein a height of the gaps varies along the flow direction.

10-14. (canceled)

15. The cell processing apparatus of claim 1, wherein a height of the gap is adjustable.

16. (canceled)

17. The cell processing apparatus of claim 1, wherein a first ridge of the plurality of ridges comprises a first ridge surface which forms a first gap with the second surface, and a second ridge of the plurality of ridges comprises a second ridge surface which forms a second gap with the second surface, and wherein the first gap has a different width than the second gap.

18-20. (canceled)

21. A system for cell processing, the system comprising:
a cell processing apparatus, comprising:
a first wall comprising a first surface;
a second wall comprising a second surface; and
a plurality of ridges connected to the first wall, wherein the plurality of ridges extends from the first surface toward the second surface, and wherein a ridge of the plurality of ridges comprises a ridge surface that forms a gap with the second surface;
a pressure source fluidically coupled to the cell processing apparatus;
one or more sensors operably coupled to the cell processing apparatus; and
a system controller, operably coupled to the pressure source and to the one or more sensors, and configured to control an operation of the pressure source based on one or more inputs from the one or more sensors.

22-24. (canceled)

25. The system of claim 21, wherein the one or more inputs comprise a pressure inside the cell processing apparatus.

26. The system of claim 21, further comprising:
an additional cell processing apparatus comprising an additional plurality of ridges, wherein a ridge of the additional plurality of ridges comprises an additional ridge surface which forms an additional gap with an additional second surface of an additional second wall of the additional cell processing apparatus, and wherein the additional gap has a different height than the gap; and
a cell sorter positioned upstream and fluidically coupled to the cell processing apparatus and the additional cell processing apparatus, such that the cell processing apparatus and the additional cell processing apparatus are configured to operate in parallel.

27. The system of claim 21, further comprising an additional cell processing apparatus, wherein the cell processing apparatus and the additional cell processing apparatus are connected in sequence.

28. The system of claim 27, further comprising an inlet fluidically coupled to and positioned between the cell processing apparatus and the additional cell processing apparatus.

29-31. (canceled)

32. A cell processing method comprising:
directing cells into a cell processing apparatus comprising:
a plurality of ridges, wherein a ridge of the plurality of ridges comprises a ridge surface that forms a gap with a surface of the cell processing apparatus,
a recovery space between two adjacent ridges of the plurality of ridges, which recovery space is configured to recover the cells after passing through the gap,
a primary inlet, positioned upstream from the plurality of ridges, and
an intermediate inlet, disposed between a pair of the plurality of ridges;
directing a reagent into the cell processing apparatus through the intermediate inlet, wherein the reagent forms a portion of a medium surrounding the cells; and
flowing the cells and the medium surrounding the cells through the gap and the recovery space to generate one or more processed cells.

33. The cell processing method of claim 32, wherein an average duration of the cells passing through the gap is less than about 1 second.

34. (canceled)

35. The cell processing method of claim 32, further comprising, sorting the cells prior to directing the cells into the cell processing apparatus.

36. The cell processing method of claim 32, further comprising, sorting the cells after flowing the cells and the medium surrounding the cells.

37. (canceled)

38. The cell processing method of claim 32, wherein a linear flow rate of the cells flowing through the cell processing apparatus is adjustable.

39. (canceled)

40. The cell processing method of claim 38, wherein the linear flow rate is adjusted in response to an input received from one or more pressure sensors.

41. The cell processing method of claim 32, further comprising reversing a direction of fluid flow of the cells through the cell processing apparatus.

42. (canceled)

43. The cell processing method of claim 32, wherein (a) comprises vibrating the cell processing apparatus.

44-45. (canceled)

46. The cell processing apparatus of claim 1, further comprising a primary inlet and an intermediate inlet, wherein:
the primary inlet is positioned upstream the plurality of ridges, and
the intermediate inlet is disposed between a pair of the plurality of ridge.

47. The cell processing method of claim 38, wherein the linear flow rate of the cells flowing through the cell processing apparatus is temporarily reversed.
